(19) United States
(12) Patent Application Publication
(10) Pub. No.: US 2016/0008391 A1
(43) Pub. Date: Jan. 14, 2016

(54) GALACTOSAMINOGALACTAN COMPRISING alpha-1-4 LINKED GALACTOSE AND alpha-1-4 LINKED N-ACETYLGALACTOSAMINE FOR USE IN THE TREATMENT OF AT LEAST ONE INFLAMMATORY DISEASE

(71) Applicants: INSTITUT PASTEUR, Paris (FR); UNIVERSITÀ DEGLI STUDI DI PERUGIA, Perugia (IT); STICHTING KATHOLIEKE UNIVERSITEIT, Nijmegen (NL)

(72) Inventors: Jean-Paul LATGE, Issy Les Moulineaux (FR); Thierry Vincent Gérard FONTAINE, Chaville (FR); Frank Leo VAN DE VEERDONK, Nijmegen (NL); Mark Sebastiaan GRESNIGT, Nijmegen (NL); Mihai NETEA, Nijmegen (NL); Leo AB JOOSTEN, Nijmegen (NL); Luigina ROMANI, Perugia (IT); Silvia BOZZA, Perugia (IT); Antonella DE LUCA, Perugia (IT)

(21) Appl. No.: 14/760,604

(22) PCT Filed: Jan. 14, 2014
(86) PCT No.: PCT/EP2014/050630

§ 371 Date: Jul. 13, 2015

(30) Foreign Application Priority Data
Jan. 14, 2013 (EP) 13305034.4

(51) Int. Cl. A61K 31/715; A61K 45/06

(52) U.S. Cl. CPC A61K 31/715; A61K 45/06

(57) ABSTRACT

A first object of the invention is galactosaminogalactan comprising α1-4 linked galactose, α1-4 linked N-acetylgalactosamine, and optionally α1-4 linked galactosamine, for use in the treatment of at least one inflammatory disease. Another object of the invention is a pharmaceutical composition comprising a galactosaminogalactan comprising α1-4 linked galactose, α1-4 linked N-acetylgalactosamine, and optionally α1-4 linked galactosamine, as defined herein, and optionally a pharmaceutically acceptable carrier. Another object of the invention is a pharmaceutical composition according to the invention, for use in the treatment of at least one inflammatory disease. Another object of the invention is an inhibitor of IL-1RA for use in the treatment of human fungal diseases. Another object of the invention is a pharmaceutical composition comprising an inhibitor of IL-1RA, and optionally a pharmaceutically acceptable carrier.

Inflammation is the body's defense reaction to injuries such as those caused by mechanical damage, infection or antigenic stimulation. An inflammatory reaction may be expressed pathologically when inflammation is induced by an inappropriate stimulus such as an autoantigen, is expressed in an exaggerated manner or persists well after the removal of the injurious agents.

While the etiology of inflammation is poorly understood, considerable information has been gained regarding the molecular aspects of inflammation (see for example Goldsby R A, Kindt T K, Osborne B A and Kuby J (2003) Immunology, 5th Edition, W.H. Freeman and Company, or Janeway C A, Travers P, Walport M, and Shlomchik M (2001) Immunobiology, 6th Edition, Garland Publishing).

Interleukin (IL)-1 was first cloned in the 1980s, and rapidly emerged as a key player in the regulation of inflammatory processes (see for reference Gabay et al., Nat Rev Rheumatol, 6: 232-241; 2010). The term IL-1 refers to two cytokines, IL-1α and IL-1β, which are encoded by two separate genes. The effects of IL-1 are tightly controlled by several naturally occurring inhibitors, such as IL-1 receptor antagonist (IL-1RA), IL-1 receptor type II (IL-1RII), and other soluble receptors.

The role of the potent proinflammatory cytokine IL-1 in disease has been shown in a broad range of diseases, including rheumatoid arthritis and autoimmune diseases. IL-1 has also been implicated in inflammatory bowel diseases (IBD). Indeed, an imbalance between proinflammatory and antiinflammatory cytokines was found for the IL-1/IL-1RA ratio in the inflamed mucosa of patients with Crohn's disease, ulcerative colitis, diverticulitis, and infectious colitis (Rogler et al.; World J Surg.; 22(4):382-9; 1998).

Nowadays, blocking of IL-1 activity (especially IL-1β) is a standard therapy for patients with autoimmune diseases or lymphomas. Recombinant IL-1RA, a potent IL-1 receptor antagonist, is approved for instance as a therapy for patients with rheumatoid arthritis, because it reduces symptoms of rheumatoid arthritis and slows the progressive joint destruction. It has also been subscribed to patients with smoldering/indolent myeloma with a high risk of progression to multiple myeloma.

The use of IL-1 receptor antagonists has been uniformly associated with beneficial effects in patients with hereditary autoinflammatory conditions associated with excessive IL-1 receptor activity (IL-1 signaling), either due to elevated IL-1 IL-1RA deficiency. Successful treatment with IL-1 blockers has also been reported in other hereditary autoinflammatory diseases, as well as in nonhereditary inflammatory diseases, such as Schnizler syndrome, systemic-onset juvenile idiopathic arthritis and adult Still disease. The role of microcrystals in the regulation of IL-1β processing and release has provided the rationale for the use of IL-1 inhibitors in crystal-induced arthritis. Finally, preliminary results indicating that IL-1 targeting is efficacious in type 2 diabetes and smoldering myeloma have further broadened the spectrum of IL-1-driven diseases.

Recombinant IL-1RA is however not devoid of sides effects, such as nausea (8%), diarrhea (7%), sinusitis (7%), flu-like syndrome (6%). The major side effect of recombinant IL-1RA is the risk of infections (40%, severe in 2%), more particularly of upper respiratory tract (13%). Moreover, recent cases of hypersensitivity to recombinant IL-1RA have been observed (Desai et al., Ann Pharmacother.; 43(5): 967-972; 2009).

There is therefore a need for alternative therapeutic compounds for the treatment for inflammatory diseases, in particular for inflammatory interleukin-1 mediated disease.

### FIGURE LEGEND

FIG. 1: Induction of IL-1RA by soluble galactosaminogalactan (GAG) is dependent on Syk and TLR3/TRIF. TNFα, IL-6, IL-8 and IL-10 concentrations in culture supernatants of human PBMCs stimulated for 24 hours with 10 μg/ml GAG and IFNγ and IL-17 concentrations after 7 days of stimulation (b) TNFα, IL-6 and IL-10 concentrations in culture supernatants of human PBMCs (n=6 donors) stimulated for 24 hours with heat inactivated A. fumigatus conidia (1×107/ml) in the presence or absence of 10 μg/ml GAG. (c,d,e) IL-17, IL-22 and IFNγ concentrations in culture supernatants of human PBMCs stimulated for 7 days with heat inactivated A. fumigatus conidia (1×107/ml) (n=10 donors for IL-17 and IL-22, n=6 donors for IFNγ) (c), IL-1β/IL-23 (50/100 ng/ml) (n=14 donors) or IL-12/IL-18 (50/100 ng/ml) (n=10 donors) in the presence or absence of GAG (10 μg/ml).

FIG. 2: GAG induces interleukin 1 receptor antagonist. (a) IL-1 bioactivity measured as IL-2 production by NOB-1 cells stimulated with 50 ng/ml IL-1β in the presence of medium of PBMCs of GAG conditioned medium (n=6 donors). (b) IL-1RA, IL-1β and IL-1α concentrations in culture supernatants of human PBMCs stimulated with for 24 hours with 10 μg/ml GAG. (c) Dose response of GAG ranging from 1 ng/ml to 10 μg/ml.

FIG. 3: Suppression of IL-17 and IL-22 by soluble galactosaminogalactan is dependent on IL-1RA. (a) IL-17, IL-22 and IFNγ concentrations in culture supernatants of PBMCs stimulated for 7 days with heat inactivated A. fumigatus conidia 1×10⁷/ml, IL-1β/IL-23 (50/100 ng/ml) or IL-12/IL-18 (50/100 ng/ml) in the presence or absence of recombinant human IL-1RA (10 ng/ml). (b) Inhibition of IL-1β/IL-23 (50/100 ng/ml) induced IL-17 and IL-22 by GAG (10 μg/ml) in human PBMCs in the presence of isotype control (10 μg/ml) or anti-IL-1RA (10 μg/ml). The IL-17 and IL-22 production by IL-1β/IL-23 in absence of GAG was set at 100%.

FIG. 4: GAG induces IL-1Ra in vivo and IL-1Ra increases susceptibility to aspergillosis. BALB/c and Il1ra^{-/-} mice were intranasally infected with Aspergillus conidia and treated with GAG (250 μg/kg intranasally) the day of and 1, 2 and 3 days post-infection). (A) Il1ra mRNA expression in mice with aspergillosis and in vitro (B) on purified cells from lungs of naive mice prestimulated with Aspergillus conidia or LPS for 1 hour and exposed to different GAG concentrations for additional 18 hours. (C) Survival, (D) fungal growth (CFU/lung, mean±SEM), (E) levels of IL-1Ra in lung homogenates of infected and GAG-treated mice, (F) BAL morphometry [% polymorphonuclear (PMNs) cells and lung histology (PAS stained sections, bars indicate 20× magnification) and (G) MPO expression in lung homogenates (assays were done a day after the last GAG treatment). (H) BAL morphometry [% PMNs or eosinophils (Eo)] and lung histology (PAS stained sections, bars indicate 20× magnification), and (I) expression of Th transcription factors and cytokines in total cells from the draining lymph nodes, by RT-PCR. Naïve, uninfected mice. None, untreated mice and/or unstimulated cells. *, p<0.05; **, p<0.01, GAG-treated vs untreated mice. (J) IL-5, IL-13 and IL-17 concentrations in culture supernatants of PBMCs pre-incubated 1 h either with medium or GAG (10 μg/ml). After washing cells were stimulated for 7 days with heat inactivated A. fumigatus conidia 1×10⁷/ml (n=4 donors). Data are represented as mean+/−SEM.

FIG. 5: Induction of IL-1RA by GAG is dependent on Syk and TLR3/TRIF. IL-1RA concentrations in culture supernatants of human PBMCs stimulated with for 24 hours with 10 μg/ml GAG in the presence or absence of (a) medium, Laminarin (50 ng/ml), anti-CR3 (10 μg/ml), (b) anti-TLR2 (10 μg/ml), bartonella LPS (20 ng/ml), or (c) syk inhibitor (10 nM). (d) Lung CFU and (e) IL-1ra mRNA expression in TLR-deficient, MyD88-deficient, and TRIF-deficient mice infected with Aspergillus conidia and treated with GAG (4 days after the infection).

FIG. 6: Measure of the inflammatory colonic pathology (FIG. 6A), damage score (FIG. 6B) and inflammatory IL-1b production and anti-inflammatory IL-10 production (FIG. 6C) in DSS-induced colitis in WT and p47phox−/− mice treated with GAG or control (no GAG).

FIG. 7: Measure of the inflammatory colonic pathology (FIG. 7A), damage score (FIG. 7B) and inflammatory IL-1b production and anti-inflammatory IL-10 production (FIG. 7C) in DSS-induced colitis in WT and p47phox−/− mice treated with Anakinra or control (no Anakinra).

FIG. 8: Gel filtration chromatography of GAG hydrolysate on G25-Sephadex column. G25I fraction was eluted from the void volume of the column. G25II was eluted before salt.

FIG. 9: IL-1Ra concentrations in culture supernatants of human PBMCs stimulated with for 24 hours with 10 μg/ml sample. Data are represented as mean+/−SEM. G25IID, G25E, G and H samples were different G25II fractions obtained from the chromatography on Sephadex G25 column. G25Ia,b,c are fractions obtained from three independent chromatographies on Sephadex G25 column. Cellulose and G25II samples were resuspended or dissolved in water. G25I fractions were dissolved in 0.25% acetic acid.

FIG. 10: ¹H-NMR spectra of the G25I fraction: NMR spectra of the fraction G25I were acquired at 338 K on a Varian Inova 500 spectrometer equipped with a triple resonance ¹H{¹³C/¹⁵N} PFG probe. Samples were solubilized in CD₃COOD 0.25% in D₂O v/v and transferred in a 5 mm NMR tube. The final concentration was about 3 mg/mL.

FIG. 11: MALDI-TOF mass spectra of oligosaccharides from G25I fraction. Pseudomolecular mass m/z=[M+Na]+ were observed.

### DESCRIPTION

A first object of the invention is galactosaminogalactan comprising α1-4 linked galactose, α1-4 linked N-acetylgalactosamine, and optionally α1-4 linked galactosamine, for use in the treatment of at least one inflammatory disease.

Thus, the invention also has for object the use of galactosaminogalactan comprising α1-4 linked galactose, α1-4 linked N-acetylgalactosamine, and optionally α1-4 linked galactosamine, for the manufacture of a medicament intended for in the treatment of at least one inflammatory disease.

Thus, the invention also has for object a method for the treatment of at least one inflammatory disease in a human subject in need thereof, comprising administering an effective amount of galactosaminogalactan comprising α1-4 linked galactose, α1-4 linked N-acetylgalactosamine, and optionally α1-4 linked galactosamine.

Another objet of the invention is a pharmaceutical composition comprising a galactosaminogalactan comprising α1-4 linked galactose, α1-4 linked N-acetylgalactosamine, and optionally α1-4 linked galactosamine as defined above, and optionally a pharmaceutically acceptable carrier.

Another object of the invention is a pharmaceutical composition according to the invention, for use in the treatment of at least one inflammatory disease.

Thus, the invention also has for object the use of a pharmaceutical composition according to the invention for the manufacture of a medicament intended for in the treatment of at least one inflammatory disease.

Thus, the invention also has for object a method for the treatment of at least one inflammatory disease in a human subject in need thereof, comprising administering an effective amount of a pharmaceutical composition according to the invention.

The inventors have further demonstrated that inhibitors of IL-1RA can efficiently prevent and/or treat human fungal diseases.

Another object of the invention is an inhibitor of IL-1RA for use in the treatment of human fungal diseases.

The object of the invention is therefore also the use of an inhibitor of IL-1RA for the manufacture of a medicament intended for the treatment of human fungal diseases.

Another object of the invention is thus a method for the treatment of human fungal diseases in a human subject in need thereof, comprising administering an effective amount of an inhibitor of IL-1RA.

Another object of the invention is a pharmaceutical composition comprising an inhibitor of IL-1RA, and optionally a pharmaceutically acceptable carrier.

Another object of the invention is thus a pharmaceutical composition an comprising an inhibitor of IL-1RA according to the invention for use in the treatment of human fungal diseases.

The object of the invention is therefore also the use of a pharmaceutical composition comprising an inhibitor of IL-1RA according to the invention for the manufacture of a medicament intended for the treatment of human fungal diseases.

Another object of the invention is thus a method for the treatment of human fungal diseases in a human subject in need thereof, comprising administering an effective amount of a pharmaceutical composition comprising an inhibitor of IL-1RA according to the invention.

### DETAILED DESCRIPTION

The inventors have found that a polysaccharide present in the cell wall of filamentous fungi can inhibit IL-1 receptor signaling. This polysaccharide, a galactosaminogalactan comprising α1-4 linked galactose, α1-4 linked N-acetylgalactosamine, and optionally α1-4 linked galactosamine, induces the expression of IL-1RA in vivo and in vitro, and therefore acts as a prodrug for IL-1RA. The galactosaminogalactan comprising α1-4 linked galactose, α1-4 linked N-acetylgalactosamine, and optionally α1-4 linked galactosamine, is a potent inhibitor of IL-1 related inflammation, and can thus efficiently be used as a treatment for inflammatory diseases. Results obtained in vivo, in an animal model for inflammatory bowel disease, show that galactosaminogalactan comprising α1-4 linked galactose, α1-4 linked N-acetylgalactosamine, and optionally α1-4 linked galactosamine, is at least as efficient, if not more efficient that commercialized recombinant IL-1RA.

A first object of the invention is thus a galactosaminogalactan comprising α1-4 linked galactose, α1-4 linked N-acetylgalactosamine, and optionally α1-4 linked galactosamine, for use in the treatment of at least one inflammatory disease.

Thus, the invention also has for object the use of a galactosaminogalactan comprising α1-4 linked galactose, α1-4 linked N-acetylgalactosamine, and optionally α1-4 linked galactosamine, for the manufacture of a medicament intended for in the treatment of at least one inflammatory disease.

Thus, the invention also has for object a method for the treatment of at least one inflammatory disease in a human subject in need thereof, comprising administering an effective amount of a galactosaminogalactan comprising α1-4 linked galactose, α1-4 linked N-acetylgalactosamine, and optionally α1-4 linked galactosamine.

By “galactosaminogalactan”, it is herein referred to a polysaccharide polymer comprising galactose and N-acetylgalactosamine residues. In a preferred embodiment, the galactosaminogalactan of the invention further comprises galactosamine residues. A “polymer” according to the invention is a compound, preferably a polysaccharide, consisting of repeating structural units. The galactosaminogalactan of the invention is thus a polysaccharide comprising galactose and N-acetylgalactosamine residues, and optionally galactosamine residues, and consisting of repeating units. Preferably, the said N-acetylgalactosamine and galactose residues, as well as the said optional galactosamine residues, are linked to the rest of the polymer through covalent bonds. More preferably, the said N-acetylgalactosamine and galactose residues are linked to the rest of the polymer through α1-4 covalent bonds. Likewise, should galactosamine residues be present in the galactosaminogalactan of the invention, these residues are linked to the rest of the polymer through α1-4 covalent bonds. The skilled person will easily realize that, in this case, the galactosaminogalactan of the invention is not branched, but linear. Hence, even more preferably, the galactosaminogalactan comprising α1-4 linked galactose and α1-4 linked N-acetylgalactosamine of the invention is a linear polysaccharide chain comprising galactose and N-acetylgalactosamine residues linked through α1-4 covalent bonds. Still more preferably, the galactosaminogalactan comprising α1-4 linked galactose, α1-4 linked N-acetylgalactosamine and α1-4 linked galactosamine of the invention is a linear polysaccharide chain comprising galactose, N-acetylgalactosamine and galactosamine residues linked through α1-4 covalent bonds.

The galactosaminogalactan of the invention can comprise a regular alternation of the galactose and N-acetylgalactosamine residues, as well as optional galactosamine residues, over the whole length of the polymer. A “regular alternation” in the context of the invention means that the repetition of a specific unit can be identified throughout the whole molecule. Alternatively, the galactose, N-acetylgalactosamine and optional galactosamine residues are randomly distributed throughout the whole polymer. By “randomly distributed”, it is herein referred to a random distribution of galactose, N-acetylgalactosamine and optional galactosamine residues on the overall length of each polymer. This definition does not exclude the existence of local patterns, i.e. that galactose, N-acetylgalactosamine and optional galactosamine be distributed according to specific patterns in some parts of the polymer. Preferably, the galactose, N-acetylgalactosamine and optional galactosamine residues are randomly distributed along the polysaccharide chain of said galactosaminogalactan. Alternatively, the galactosaminogalactan of the invention preferably comprises at least one monomer having the formula:

(GalNAc)ₙ,

wherein n is an integer comprised between 5 and 12.

More preferably, n is comprised between 6 and 12; still more preferably, between 7 and 12; still more preferably, between 8 and 12; still more preferably, between 9 and 12; still more preferably, between 10 and 12; or still more preferably, between 11 and 12.

In a preferred embodiment, said galactosaminogalactan has an average ratio of galactose/(N-acetylgalactosamine+galactosamine) of between 1/99 and 99/1. More preferably, said galactosaminogalactan has an average ratio of galactose/(N-acetylgalactosamine+galactosamine) of between 40/60 and 60/40. Even more preferably, said galactosaminogalactan polymer has an average ratio of galactose/(N-acetylgalactosamine+galactosamine) of around 50/50. By “ratio of galactose/(N-acetylgalactosamine+galactosamine)”, it is herein referred to the molar ratio of galactose/(N-acetylgalactosamine+galactosamine). By “N-acetylgalactosamine+galactosamine”, it is herein referred to the total of all glucosamine forms, i.e. both N-acetylated and N-deacetylated. In a first preferred embodiment, the galactosaminogalactan polymer of the invention does not comprise any detectable N-deacetylated galactosamine. In that case, the N-acetylgalactosamine+galactosamine fraction corresponds solely to the N-acetylgalactosamine form. In another preferred embodiment, the galactosaminogalactan polymer of the invention comprises N-deacetylated galactosamine residues. In a further preferred embodiment, the ratio of the galactosamine/(N-acetylgalactosamine+galactosamine) is at least 2%. In other words, according to this embodiment, the fraction of the total galactosamine which is N-deacetylated is at least 2%. The average ratio of galactose/(N-acetylgalactosamine+galactosamine) can easily be determined by gas liquid chromatography analysis, NMR spectroscopy, or any other technique known in the art for identifying and quantifying the constituents of a polysaccharide chain.

According to a preferred embodiment, the molecular weight of the galactosaminogalactan of the invention is at least 1 kDa. In a further preferred embodiment, the said molecular weight is comprised between 1 and 1000 kDa. In another further preferred embodiment, said molecular weight is comprised between 10 and 1000 kDa. Still more preferably, said galactosaminogalactan has an average molecular weight of 100 kDa. In yet another further preferred embodiment, the molecular weight of the galactosaminogalactan of the invention is comprised between 1 and 10 kDa; more preferably, between 1 and 5 kDa; still more preferably, between 1 and 3 kDa. The molecular weight of the galactosaminogalactan polymer can easily be estimated by any technique known to the person skilled in the art, such as e.g. size-exclusion chromatography (also called gel permeation chromatography or gel filtration chromatography). By the term “average molecular weight” of the galactosaminogalactan, it is herein referred to the average of the molecular weights of a population of galactosaminogalactan molecules.

In the most preferred embodiment of the invention, the said galactosaminogalactan polymer has a molecular weight comprised between 1 and 3 kDa, and comprises between 5 and 12, between 6 and 12, between 7 and 12, between 8 and 12, between 9 and 12, between 10 and 12, or between 11 and 12 α1-4 linked N-acetylgalactosamine residues.

In a particular embodiment of the invention, said galactosaminogalactan is soluble in urea. Preferably, said galactosaminogalactan is soluble in 8M urea.

The galactosaminogalactan of the invention can be obtained by chemical synthesis. In this regard, the said galactosaminogalactan can be synthesized by any technique known to the person of skills in the art. Alternatively, the said galactosaminogalactan is a naturally occurring polymer, e.g. a polymer obtained from biological sources.

Polymers containing galactosamine have been described in various biological organisms. In particular, they are found in the cell wall of filamentous fungi, including Penicillium frequentans (Guerrero et al., Microbiologia, 4(1):39-46, 1988), Aspergillus parasiticus (Ruperez et al., Trans Br Mycol Soc, 77(3): 621-625, 1981), Neurospora crassa, Rhizopus, Aspergillus niger (Bardalaye & Nordin, J Bacteriol, 125(2): 655-669, 1976), Aspergillus fumigatus (Fontaine et al., PLoS Pathog., 7(11):e1002372, 2011; Gravelat, PLoS Pathog, 9(8): e1003575, 2013; Sheppard, Curr Opin Microbiol, 14(4): 375-379, 2011). In particular, it was previously shown that Aspergillus fumigatus comprises a cell wall component that is shed into the environment during Aspergillus growth. This cell wall component is a linear heterogeneous galactosaminogalactan composed of α1-4 linked galactose and α1-4 linked N-acetylgalactosamine residues (Fontaine et al., PLoS Pathog., 7(11):e1002372, 2011). The galactosaminogalactan of the invention can hence be isolated from a culture supernatant of Aspergillus fumigatus.

In a particular embodiment of the invention, said galactosaminogalactan is obtained from Aspergillus fumigatus.

By “Aspergillus fumigatus”, it is herein referred to a filamentous fungus of the genus Aspergillus, which genomic sequence was published in Nierman et al. (Nature 438, 1151-1156, 2005). Aspergillus fumigatus is a ubiquitous fungus that usually develops on decaying plant material and in soil. Therefore, the skilled person can use any isolate obtained from such samples. Any known strain or isolate of Aspergillus fumigatus can be used in the context of the invention. The skilled person can also use for example the reference strain CBS 144-89.

Preferably, the galactosaminogalactan of the invention is obtained from the mycelium. By “mycelium”, it is herein referred to the vegetative part of a filamentous fungus, preferably Aspergillus fumigatus, consisting of a mass of long, branching filamentous structures called hyphae. Various methods are known for obtaining the mycelium of Aspergillus fumigatus; Aspergillus fumigatus mycelium can thus easily be obtained from an Aspergillus fumigatus culture for example by filtration, preferably under vacuum.

In a particular embodiment of the invention, said galactosaminogalactan is obtained from Aspergillus fumigatus by a process comprising the culture of Aspergillus fumigatus, advantageously of Aspergillus fumigatus mycelium.

The skilled person may use any technique known in the art in order to obtain the galactosaminogalactan of the invention from Aspergillus fumigatus. Advantageously, the inventors have found that galactosaminogalactan can be extracted from the total fungal carbohydrates by a urea extraction step.

In a particular embodiment of the invention, said galactosaminogalactan is thus obtained from Aspergillus fumigatus by a process comprising an extraction step by urea.

By “extraction by urea” or “urea extraction”, it is herein referred to a step of treating a product with a urea solution in view of partial or total solubilisation of said product, and recovery of the soluble fraction. By “soluble fraction obtained from extraction by urea” or “urea-soluble fraction”, it is herein referred to the soluble fraction that can be recovered by the skilled person after urea extraction. Preferably, the extraction step in urea is performed in 8 M urea.

Thus, according to the present embodiment, said galactosaminogalactan is obtained from Aspergillus fumigatus by a process comprising a step of treatment with urea and recovery of the urea-soluble fraction. Preferably, said galactosaminogalactan is obtained from a mycelium of Aspergillus fumigatus by a process comprising a step of treatment with urea and recovery of the urea-soluble fraction.

In a preferred embodiment of the invention, said galactosaminogalactan is obtained from Aspergillus fumigatus by a process comprising a step of growing Aspergillus fumigatus and a step of treating said Aspergillus fumigatus culture with urea and recovering the urea-soluble fraction. Aspergillus fumigatus has been a model organism for years (Latgé, Clin Microbiol Rev, 12(2): 310-350, 1999). Methods for growing this fungus are thus well known to the skilled person and do not require to be thoroughly explained herein. For example, methods for isolation and culture of Aspergillus fumigatus are described in Nieminen et al. (Appl Environ Microbiol.; 68(10):4871-5; 2002). An example of such a method is detailed in the experimental examples section. More preferably, Aspergillus fumigatus is grown for at least 30 hours. Still more preferably, Aspergillus fumigatus is grown for at least 50 hour.

Preferably, the culture of Aspergillus fumigatus is filtered prior to the extraction step by urea. More preferably, the culture of Aspergillus fumigatus is filtered under vacuum prior to the extraction step by urea.

In order to increase the yield and purity of the galactosaminogalactan, it is advantageous to deplete the culture of Aspergillus fumigatus from glycoproteins and galactomannans by incubation in a NaCl solution, prior to the extraction step in urea. By “incubation in NaCl”, it is herein referred to incubation in an NaCl solution. The insoluble fraction, or NaCl-insoluble fraction, corresponds to all the material which cannot be solubilized in the NaCl solution. The inventors have found that said NaCl-insoluble fraction comprises the galactosaminogalactan of the invention, while most of the glycoproteins and galactomannans remain soluble in the NaCl solution.

Therefore, in a preferred embodiment, the process of preparation of said galactosaminogalactan of the invention comprises a step of incubation in NaCl, advantageously prior to the extraction step in urea. According to this embodiment, the said galactosaminogalactan is obtained from Aspergillus fumigatus by a process comprising a step of growing Aspergillus fumigatus, a step of incubating said Aspergillus fumigatus in NaCl, and a step of treating the NaCl-insoluble fraction of the previous step with urea and recovering the urea-soluble fraction.

Preferably, the NaCl solution of the invention has a concentration comprised between 100 and 200 mM. More preferably, the concentration of the NaCl solution is comprised between 120 and 170 mM. Even more preferably, the concentration of the NaCl solution is of 150 mM. Preferably, the culture of Aspergillus fumigatus is filtered prior to the incubation in NaCl. More preferably, the culture of Aspergillus fumigatus is filtered under vacuum prior to the incubation in NaCl.

In order to further increase the yield and purity of the galactosaminogalactan, the skilled person can additionally precipitate the culture of Aspergillus fumigatus in an alcohol solution, advantageously prior to the extraction step in urea.

Preferably, the process of the invention comprises a step of alcohol precipitation, advantageously prior to the extraction step in urea. More preferably, the step of alcohol precipitation is performed prior to incubation in a NaCl solution.

By “alcohol precipitation”, it is herein referred to the incubation of a compound, preferably a polysaccharide, in an alcohol solution and recovery of the precipitate. The “precipitate obtained from alcohol precipitation” corresponds to the insoluble fraction that can be recovered by the skilled person after alcohol precipitation. By “alcohol solution”, it is herein referred to any aqueous solution comprising an alcohol, i.e. an organic compound comprising a hydroxyl functional group (—OH) bound to a carbon atom. Preferably, the alcohol compound is chosen from ethanol, methanol, isopropyl alcohol and butyl alcohol. More preferably, the alcohol compound is ethanol. Preferably, the alcohol solution comprises more than 50, 60, 70, 80, or 90% of alcohol by weight compared to the total weight of the solution. More preferably, the alcohol solution comprises between 60 and 80% of alcohol by weight compared to the total weight of the solution. Even more preferably, the alcohol solution comprises 70% of alcohol by weight compared to the total weight of the solution.

Preferably, the culture of Aspergillus fumigatus is filtered prior to the alcohol precipitation. More preferably, the culture of Aspergillus fumigatus is filtered under vacuum prior to the alcohol precipitation.

In a particular embodiment of the invention, said galactosaminogalactan is obtained from Aspergillus fumigatus by a process comprising the following steps:
a) culture of Aspergillus fumigatus;
b) alcoholic precipitation of the culture obtained from step a);
c) incubation of the precipitate obtained from step b) in NaCl; and
d) extraction of the insoluble fraction obtained from step c) by urea.

In order to obtain the fraction displaying the specific activity, it is possible to subject the galactosaminogalactan to a mild acid hydrolysis. Mild acid hydrolysis is a widely used technique for preparing oligosaccharides fractions from polysaccharide preparations. The skilled person will, for example, refer to Fontaine et al. (PLoS Pathog., 7(11):e1002372, 2011) for an example of mild hydrolysis conditions which yield oligosaccharides out of the galactosaminogalactan fraction.

Therefore, in a preferred embodiment of the invention, the process for producing galactosaminogalactan further comprises a step of mild acid hydrolysis of the product obtained in step d).

As explained above, it has been surprisingly found that the galactosaminogalactan of the invention is capable of inhibiting IL-1-mediated inflammation. Additionally, the inventors have found that the galactosaminogalactan of the invention is capable of inducing the expression of IL-1RA both in vitro and in vivo.

The interleukin-1 receptor antagonist (IL-1RA) (NCBI ref.: NP_{—}000568.1) is a protein that in humans is encoded by the IL1RN gene (NCBI ref.: NG_{—}021240.1). The IL-1RA protein binds non-productively to the cell surface interleukin-1 receptor (IL-1R), i.e. without activating the IL-1R receptor, while it inhibits the binding of IL1-alpha and IL1-beta. IL-1RA is used in the treatment of inflammatory diseases, among which rheumatoid arthritis, an autoimmune disease in which IL-1 plays a key role. It is commercially produced as anakinra, which is a human recombinant form of IL-1RA.

In a particular embodiment of the invention, said galactosaminogalactan induces the expression of IL-1RA. The induction of IL-1RA may be determined by measuring the concentration of IL-1RA at the transcript level, and/or at the protein level. Without being bound by theory, it is thought that the increase in the IL-1RA protein levels resulting from the increased gene expression, leads to an inhibition of the IL-1R receptor, thus blocking the activation of the IL-1 mediated inflammation pathway.

According to this embodiment, administration of the galactosaminogalactan of the invention to a subject results in an induction of the expression of IL-1RA. This induction can be detected by taking a biological sample from the said subject, measuring the level of expression of IL-1RA, and comparing said level to a control. Said control is advantageously obtained from a subject to whom said galactosaminogalactan has not been administered. This control can be obtained any subject from the general population who has not received the galactosaminogalactan administration. Preferably, this control is obtained from the subject who is treated, prior to the administration of the said galactosaminogalactan.

A “biological sample” may be any sample that may be taken from a subject. Such a sample must allow for the determination of the expression levels of the IL-1RA protein or transcript. More specifically, a biological sample is a subset of biological tissues from an organism, its cells or component parts (e.g., body fluids, including but not limited to, blood, mucus, lymphatic fluid, synovial fluid, cerebrospinal fluid, saliva, amniotic fluid, amniotic cord blood, urine, vaginal fluid and semen). A biological sample further refers to a homogenate, lysate or extract prepared from a subset of biological tissues from an organism, its cells or component parts, or a fraction or portion thereof, including but not limited to, for example, blood, blood cells, plasma, serum, spinal fluid, lymph fluid, the external sections of the skin, respiratory, intestinal, and genitourinary tracts, tears, saliva, milk, organs. Most often, the sample has been removed from a subject. Preferably, the biological sample is blood.

The method for detecting IL-1RA expression according to the invention may thus comprise a preliminary step, between the taking of the sample from the patient and the measuring of the concentration of IL-1RA at the transcript level, and/or at the protein level, said step corresponding to the transformation of the biological sample into a mRNA (or corresponding cDNA) sample or into a protein sample, which is then ready to use for in vitro measuring of genes expression level or protein level. Preparation or extraction of mRNA (as well as retrotranscription into cDNA) or proteins from a tissue sample is only routine procedure well known to those skilled in the art.

Once a ready-to-use mRNA (or corresponding cDNA) or protein sample is available, the measure of IL-1RA gene expression levels may be performed, depending on the type of transformation and the available ready-to-use sample, either at the mRNA (i.e. based on the mRNA content of the sample) or at the protein level (i.e. based on the protein content of the sample).

By “measuring the concentration of IL-1RA at the transcript level” it is herein referred to measuring the quantity and/or the concentration of the transcript IL-1RA (NCBI ref.: NM_{—}000577.4), which is the transcript of the IL1RN gene (NCBI ref.: NG_{—}021240.1). Measuring the concentration of IL-1RA at the transcript level can be done with any technique used to measure and quantify nucleic acids.

When expression levels are thus measured at the mRNA level, it may be notably performed using well known technologies such as quantitative PCR or nucleic acid microarray technologies (including cDNA and oligonucleotide microarrays). These technologies are now used routinely by those skilled in the art and thus do not need to be detailed here. Alternatively, any known or future technology permitting to assess genes expression levels based on mRNA contents may be used. For instance, tissue microarrays coupled to fluorescent in situ hybridization may be used. Tissue microarrays (also known as TMAs) consist of paraffin blocks in which up to 1000 separate tissue cores are assembled in array fashion to allow multiplex histological analysis. In the tissue microarray technique, a hollow needle is used to remove tissue cores as small as 0.6 mm in diameter from regions of interest in paraffin-embedded tissues such as clinical biopsies or tumor samples. These tissue cores are then inserted in a recipient paraffin block in a precisely spaced, array pattern. Sections from this block are cut using a microtome, mounted on a microscope slide and then analyzed by any method of standard histological analysis. Each microarray block can be cut into 100-500 sections, which can be subjected to independent tests. Tests commonly employed in tissue microarray include immunohistochemistry, and fluorescent in situ hybridization. For analysis at the mRNA level, tissue microarray technology may be coupled to fluorescent in situ hybridization.

In a preferred embodiment, the concentration of IL-1RA at the transcript level is measured using quantitative PCR. Quantitative, or real-time, PCR is a well-known and easily available technology for those skilled in the art and does not need a precise description.

By “measuring the concentration of IL-1RA at the protein level” it is herein referred to measuring the quantity and/or the concentration of the protein IL-1RA (NCBI ref.: NP_{—}000568.1).

When IL-1RA expression levels are measured at the protein level, it may be notably performed using specific antibodies, in particular using well known technologies such as cell membrane staining using biotinylation or other equivalent techniques followed by immunoprecipitation with specific antibodies, western blot, ELISA or ELISPOT, antibodies microarrays, or tissue microarrays coupled to immunohistochemistry. Other suitable techniques include FRET or BRET, single cell microscopic or histochemistry methods using single or multiple excitation wavelength and applying any of the adapted optical methods, such as electrochemical methods (voltametry and amperometry techniques), atomic force microscopy, and radio frequency methods, e.g. multipolar resonance spectroscopy, confocal and non-confocal, detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry), cell ELISA, flow cytometry, radioisotopic, magnetic resonance imaging, analysis by mass spectrometry (MS), tandem mass spectrometry (MS-MS), MS 3; matrix-assisted laser desorption/ionization time-of-flight (MALDI-TOF) mass spectrometry; polyacrylamide gel electrophoresis (SDS-PAGE); HPLC-Mass Spectroscopy; Liquid Chromatography/Mass Spectrometry/Mass Spectrometry (LC-MS/MS)). All these techniques are well known in the art and need not be further detailed here.

An “antibody specific for IL-1RA” is an immunoglobulin molecule or a derivative thereof, which is capable of binding selectively and reversibly to IL-1RA. By “selectively binds” it is herein referred to the ability of antibodies to preferentially bind to IL-1RA, in comparison with other antigens. Generally, the interaction of an antibody with an antigen, such as IL-1RA, can be characterized in terms of a binding affinity, which is commonly expressed by the affinity constant. The affinity constant (also known as the association constant), Ka, is a numerical constant used to describe the binding affinity of two molecules at equilibrium. Preferably, an antibody will be said to be specific for IL-1RA when the binding affinity of said antibody for said IL-1RA will be superior to the binding affinity of the same antibody for unrelated antigens. The binding affinity can be measured using a variety of methods known to those skilled in the art including immunoblotting, immunoprecipitation analyzes, Radio-Immuno Assays, ELISAs, assays of antibodies by immunofluorescence microscopy, surface plasmon resonance (BiaCORE). Preferably, the affinity of an antibody specific for an antigen has an affinity constant of between about 10³M⁻¹ and about 10¹²M⁻¹ for said antigen.

Techniques to produce polyclonal or monoclonal antibodies specific for IL-1RA and/or for fragments thereof are well known to those skilled in the art and need not be described in detail herein. Polyclonal antibodies can be obtained by immunization, possibly by multiple immunizations, of an animal with said IL-1RA, followed by recovery of serum from said animal and purification of the desired antibodies, in particular by affinity chromatography using the polypeptide used for the immunization. Monoclonal antibodies may be obtained by the hybridoma method described in Köhler et al. (Nature, 1975, 256 (5517): 495-497). Methods for preparation and use of antibodies, and the assays mentioned herein before are described in Harlow, E. and Lane, D. Antibodies: A Laboratory Manual, (1988), Cold Spring Harbor Laboratory Press. Alternatively, it is possible to use commercially available anti-IL-1RA antibodies, such as for example the monoclonal mouse IgG2A Clone#10309 (catalog number: MAB601) from R&D system, or the polyclonal goat IgG (catalog number: AF-201-NA) from R&D system.

Since galactosaminogalactan induces the expression of IL-1RA, it is most useful in the treatment of diseases associated with so-called IL-1 diseases. Thus, in a particular embodiment of the invention, said inflammatory disease is an interleukin-1 mediated disease.

By “interleukin-l mediated disease” it is herein referred to a spontaneous or experimental disease or medical condition that is associated with elevated levels of IL-1 in bodily fluids or tissue or if cells or tissues taken from the body produce elevated levels of IL-1 in culture. In many cases, such interleukin-l mediated diseases are also recognized by the following additional two conditions: (1) pathological findings associated with the disease or medical condition can be mimicked experimentally in animals by the administration of IS and (2) the pathology induced in experimental animal models of the disease or medical condition can be inhibited or abolished by treatment with agents which inhibit the action of IL-1. In most interleukin-l mediated diseases at least two of the three conditions are met, and in many interleukin-l mediated diseases all three conditions are met.

A non-exclusive list of acute and chronic interleukin-l (IL-l)-mediated inflammatory diseases includes but is not limited to the following: acute pancreatitis; ALS; Alzheimer's disease; cachexia/anorexia; asthma; atherosclerosis; chronic fatigue syndrome, fever; diabetes (e.g., insulin diabetes); glomerulonephritis; graft versus host rejection; hemorrhagic shock; hyperalgesia, inflammatory bowel diseases; inflammatory conditions of a joint, including osteoarthritis, psoriatic arthritis and rheumatoid arthritis; ischemic injury, including cerebral ischemia (e.g., brain injury as a result of trauma, epilepsy, hemorrhage or stroke, each of which may lead to neurodegeneration); lung diseases (e.g., ARDS); multiple myeloma; multiple sclerosis; myelogenous (e.g., AML and CML) and other leukemias; myopathies (e.g., muscle protein metabolism, esp. in sepsis); osteoporosis; Parkinson's disease; pain; pre-term labor; psoriasis; reperfusion injury; septic shock; side effects from radiation therapy, temporal mandibular joint disease, tumor metastasis; or an inflammatory condition resulting from strain, sprain, cartilage damage, trauma, orthopedic surgery, infection or other disease processes.

In a particular embodiment of the invention, said inflammatory disease is chosen from acute pancreatitis, ALS, Alzheimer's disease, cachexia/anorexia, asthma, atherosclerosis, chronic fatigue syndrome, fever, insulin diabetes, glomerulonephritis, graft versus host rejection, hemorrhagic shock, hyperalgesia, inflammatory bowel diseases, inflammatory conditions of a joint, including osteoarthritis, psoriatic arthritis and rheumatoid arthritis, ischemic injury, including cerebral ischemia (e.g., brain injury as a result of trauma, epilepsy, hemorrhage or stroke, each of which may lead to neurodegeneration), lung diseases (e.g., ARDS), multiple myeloma, multiple sclerosis, myelogenous (e.g., AML and CML) and other leukemias, myopathies (e.g., muscle protein metabolism, esp. in sepsis), osteoporosis, Parkinson's disease, pain, pre-term labor, psoriasis, reperfusion injury, septic shock, side effects from radiation therapy, temporal mandibular joint disease, tumor metastasis, or an inflammatory condition resulting from strain, sprain, cartilage damage, trauma, orthopedic surgery, infection.

In a particular embodiment of the invention, said inflammatory disease is chosen from inflammatory bowel diseases.

In a particular embodiment of the invention, said inflammatory bowel disease is chosen from Crohn's disease, ulcerative colitis, diverticulitis, and infectious colitis.

In a particular embodiment of the invention, said inflammatory disease is chosen from rheumatoid arthritis, osteoarthritis, and other inflammatory conditions resulting from strain, sprain, trauma, infection, cartilage damage or orthopedic surgery.

In a particular embodiment of the invention, said inflammatory disease is chosen from inflammatory joint disease, multiple sclerosis, leukemia, ischemic injury, or reperfusion injury.

Further, the galactosaminogalactan of the invention may be administered via topical, enteral or parenteral administration including, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal, intraventricular and intrasternal injection and infusion. The galactosaminogalactan of the invention may also be administered via oral administration or be administered through mucus membranes, that is, intranasally, sublingually, buccally or rectally for systemic delivery.

In a specific embodiment, for the treatment of inflammatory bowel diseases for example, it is preferred that the galactosaminogalactan of the invention be administered via oral administration or be administered through mucus membranes (mucosa).

In a specific embodiment, for the treatment of rheumatoid arthritis and osteoarthritis for example, it is preferred that the galactosaminogalactan of the invention be administered via intraarticular, subcutaneous, intramuscular or intravenous injection.

In a specific embodiment, for the treatment of brain injury as a result of trauma, epilepsy, hemorrhage or stroke, or for the treatment of graft-versus-host disease for example, it is preferred that the galactosaminogalactan of the invention be administered via intraventricular or intravenous injection.

Regardless of the manner of administration, the treatment of IL-l-mediated disease requires a dose or total dose regimen of the galactosaminogalactan of effective amounts, i.e., effective to prevent, reduce or alleviate symptoms of the disease, such as to counteract progressive mucosa destruction in the case of inflammatory bowel diseases.

The effective amount is calculated according to the general criteria in the medical field, such as the approximate body weight or surface area of the patient, the disease or condition to be treated or prevented, the severity of the disease, the route of administration, and the age, sex and medical condition of the patient. Further refinement of the calculations necessary to determine the appropriate dosage for treatment is routinely made by those skilled in the art.

The frequency of dosing depends on the disease and condition of the patient, as well as the pharmacokinetic parameters of the galactosaminogalactan used in the formulation, and the route of administration. The galactosaminogalactan may be administered once, or in cases of severe and prolonged disorders, administered daily in less frequent doses or administered with an initial bolus dose followed by a continuous dose or sustained delivery.

In order to facilitate their administration, it is advantageous to formulate the compounds of the invention into compositions.

In another aspect, the invention also provides a pharmaceutical composition comprising a galactosaminogalactan comprising α1-4 linked galactose and α1-4 linked N-acetylgalactosamine above, and optionally a pharmaceutically acceptable carrier.

A “pharmaceutically acceptable carrier” according to the invention is a compound, or a combination of compounds, contained in a pharmaceutical composition, that does not cause secondary reactions and that, for example, facilitates administration of the active compounds, increases its lifespan and/or effectiveness in the organism, increases its solubility in solution or improves its storage. Such pharmaceutical carriers are well-known and will be adapted by a person skilled in the art according to the nature and the administration route of the active compounds selected.

In an embodiment, the composition of the invention is preferably formulated for oral administration.

For oral administration, the composition of the invention is preferably formulated in the form of dosage units, such as ingestible tablets, buccal tablets, capsules, or in the form of elixirs, suspensions, syrups, wafers and the like.

The dosage unit can further contain the following: a binder such as gum tragacanth, acacia, corn starch or gelatin, excipients such as dicalcium phosphate, a disintegrating agent such as corn starch, alginic acid and the like, a lubricant such as magnesium stearate, a sweetening agent such as sucrose, lactose or saccharin, or a flavoring agent such as peppermint, oil of wintergreen or cherry or orange flavoring. Various other materials can be present as a coating or to otherwise modify the physical form of the dosage unit. For instance, tablets, pills or capsules can be coated with shellac, sugar or both.

In another embodiment, the composition of the invention is formulated for administration through mucosa.

Those of ordinary skill in the clinical arts will be familiar with formulations and vehicles for drug delivery into mucosa. Useful references in this regard are Chien (Novel Drug delivery system, Chapters 3 through 6 and 9, Marcel Dekker, 1992), and Pharmaceutical Dosage Forms and Drug Delivery Systems (ANSEL et al., 1994, WILLIAMS a WILKINS).

Administration through mucosa can be obtained by formulating the composition of the invention into sprays and the like (e.g., aerosol spray or pump spray and the like), solutions, or as gels. When formulated for administration through mucosa, the composition of the invention comprises a vehicle selected in the group comprising solutions, emulsions, microemulsions, oil-in-water emulsions, anhydrous lipids and oil-in-water emulsions, other types of emulsions.

In another embodiment, the composition of the invention is preferably formulated for parenteral administration.

Parenteral administration can be obtained by formulating the composition of the invention into injectable formulations. Injectable formulations frequently comprise mixtures of water, organic solvents and surfactants.

Preferably, the composition of the invention comprises an effective amount of galactosaminogalactan, as defined above.

Pharmaceutical compositions of the present invention may be administered with other therapeutics suitable for the indication being treated, such as for example a second anti-inflammatory drug. The galactosaminogalactan or pharmaceutical composition comprising thereof of the invention and any of one or more additional anti-inflammatory drugs may be administered separately or in combination. Information regarding anti-inflammatory drugs can be found in “The Merck Manual of Diagnosis and Therapy” (19^{th} edition, Merck, Sharp a Dohme Research Laboratories, Merck Et Co., Rahway, N.J. (2011)).

Present treatment of IL-1 mediated diseases, as defined hereabove, including acute and chronic inflammation such as inflammatory conditions of a joint (e.g., rheumatoid arthritis) includes first line drugs for control of pain and inflammation, classified as non-steroidal anti-inflammatory drugs (NSAIDs), corticosteroids, slow acting anti-rheumatic drugs (SAARDs), or disease-modifying antiartrithic drugs (DMARDs).

In a particular embodiment of the invention, said pharmaceutical composition comprises at least a second anti-inflammatory compound chosen from non-steroidal anti-inflammatory drugs (NSAIDs), corticosteroids, slow acting anti-rheumatic drugs (SAARDs), or disease-modifying antiarthritic drugs (DMARDs).

Non-steroidal anti-inflammatory drugs (NSAIDs) include aspirin, ibuprofen, and naproxen. NSAIDs can be characterized into nine groups: (1) salicylic acid derivatives; (2) propionic acid derivatives; (3) acetic acid derivatives; (4) fenamic acid derivatives; (5) carboxylic acid derivatives; (6) butyric acid derivatives; (7) oxicams; (8) pyrazoles and (9) pyrazolones.

According to the invention, salicylic acid derivatives include prodrug esters and pharmaceutically acceptable salts thereof comprise: acetaminosalol, aloxiprin, aspirin, benorylate, bromosaligenin, calcium acetylsalicylate, choline magnesium trisalicylate diflusinal, etersalate, fendosal, gentisic acid, glycol salicylate, imidazole salicylate, lysine acetylsalicylate, mesalamine, morpholine salicylate, l-naphthyl salicylate, olsalazine, parsalmide, phenyl acetylsalicylate, phenyl salicylate, salacetamide, salicylamide O-acetic acid, salsalate and sulfasalazine.

According to the invention, propionic acid derivatives include alminoprofen, benoxaprofen, bucloxic acid, carprofen, dexindoprofen, fenoprofen, flunoxaprofen, fluprofen, flurbiprofen, furcloprofen, ibuprofen, ibuprofen aluminum, ibuproxam, indoprofen, isoprofen, ketoprofen, loxoprofen, miroprofen, naproxen, oxaprozin, piketoprofen, pimeprofen, pirprofen, pranoprofen, protizinic acid, pyridoxiprofen, suprofen, tiaprofenic acid and tioxaprofen.

According to the invention, acetic acid derivatives include acemetacin, alclofenac, amfenac, bufexamac, cinmetacin, clopirac, delmetacin, diclofenac sodium, etodolac, felbinac, fenclofenac, fenclorac, fenclozic acid, fentiazac, furofenac, glucametacin, ibufenac, indomethacin, isofezolac, isoxepac, lonazolac, metiazinic acid, oxametacin, oxpinac, pimetacin, proglumetacin, sulindac, talmetacin, tiaramide, tiopinac, tolmetin, zidometacin and zomepirac.

According to the invention, fenamic acid derivatives include enfenamic acid, etofenamate, flufenamic acid, isonixin, meclofenamic acid, meclofenamate sodium, medofenamic acid, mefanamic acid, niflumic acid, talniflumate, terofenamate, tolfenamic acid and ufenamate.

According to the invention, carboxylic acid derivatives include clidanac, diflunisal, flufenisal, inoridine, ketorolac and tinoridine.

According to the invention, butyric acid derivatives include bumadizon, butibufen, fenbufen and xenbucin.

According to the invention, oxicams include droxicam, enolicam, isoxicam, piroxicam, sudoxicam, tenoxicam and 4-hydroxyl-1,2-benzothiazine 1,1-dioxide 4-(N-phenyl)-carboxamide.

According to the invention, pyrazoles include difenamizole and epirizole.

According to the invention, pyrazolones include apazone, azapropazone, benzpiperylon, feprazone, mofebutazone, morazone, oxyphenbutazone, phenylbutazone, pipebuzone, propylphenazone, ramifenazone, suxibuzone and thiazolinobutazone.

By corticosteroids, it is herein referred to analogues of steroid hormones naturally produced in the adrenal cortex of vertebrates, and that are chemically synthesized. According to the invention, corticosteroids include hydrocortisone and compounds which are derived from hydrocortisone, such as 21-acetoxypregnenolone, alclomerasone, algestone, amcinonide, beclomethasone, betamethasone, betamethasone valerate, budesonide, chloroprednisone, clobetasol, clobetasol propionate, clobetasone, clobetasone butyrate, clocortolone, cloprednol, corticosterone, cortisone, cortivazol, deflazacon, desonide, desoximerasone, dexamethasone, diflorasone, diflucortolone, difluprednate, enoxolone, fluazacort, flucloronide, flumethasone, flumethasone pivalate, flunisolide, flucinolone acetonide, fluocinonide, fluorocinolone acetonide, fluocortin butyl, fluocortolone, fluorocortolone hexanoate, diflucortolone valerate, fluorometholone, fluperolone acetate, fluprednidene acetate, fluprednisolone, flurandenolide, formocortal, halcinonide, halometasone, halopredone acetate, hydrocortamate, hydrocortisone, hydrocortisone acetate, hydrocortisone butyrate, hydrocortisone phosphate, hydrocortisone 21-sodium succinate, hydrocortisone tebutate, mazipredone, medrysone, meprednisone, methylprednicolone, mometasone furoate, paramethasone, prednicarbate, prednisolone, prednisolone 21-diedryaminoacetate, prednisolone sodium phosphate, prednisolone sodium succinate, prednisolone sodium 21-m-sulfobenzoate, prednisolone sodium 21-stearoglycolate, prednisolone tebutate, prednisolone 21-trimethylacetate, prednisone, prednival, prednylidene, prednylidene 21-diethylaminoacetate, tixocortol, triamcinolone, triamcinolone acetonide, triamcinolone benetonide and triamcinolone hexacetonide.

Slow acting anti-rheumatic drugs (SAARDs) have also been called Disease-modifying antiarthritic drugs (DMARDs) in the literature and include cyclosporine, azulfidine (sulfasalazine), methotrexate, imuran (azathioprine), cytoxan (cyclophosphamide), actemra, cimzia, enbrel, humira, kineret, orencia, remicade, rituxan, and simponi, Adalimumab, Azathioprine, Chloroquine, hydroxychloroquine, cyclosporin (Cyclosporin A), D-penicillamine, Etanercept, Golimumab, sodium aurothiomalate, auranofin, Infliximab, Leflunomide, Minocycline, Rituximab.

It is thus another object of the present invention to provide a pharmaceutical composition according to the invention, for use in the treatment of at least one inflammatory disease.

Thus, the invention also has for object the use of a pharmaceutical composition according to the invention for the manufacture of a medicament intended for in the treatment of at least one inflammatory disease.

Thus, the invention also has for object a method for the treatment of at least one inflammatory disease in a human subject in need thereof, comprising administering an effective amount of a pharmaceutical composition according to the invention.

In a particular embodiment of the invention, said inflammatory disease is an interleukin-1 mediated disease.

In a particular embodiment of the invention, said inflammatory disease is chosen from acute pancreatitis, ALS, Alzheimer's disease, cachexia/anorexia, asthma, atherosclerosis, chronic fatigue syndrome, fever, insulin diabetes, glomerulonephritis, graft versus host rejection, hemohorragic shock, hyperalgesia, inflammatory bowel diseases, inflammatory conditions of a joint, including osteoarthritis, psoriatic arthritis and rheumatoid arthritis, ischemic injury, including cerebral ischemia (e.g., brain injury as a result of trauma, epilepsy, hemorrhage or stroke, each of which may lead to neurodegeneration), lung diseases (e.g., ARDS), multiple myeloma, multiple sclerosis, myelogenous (e.g., AML and CML) and other leukemias, myopathies (e.g., muscle protein metabolism, esp. in sepsis), osteoporosis, Parkinson's disease, pain, pre-term labor, psoriasis, reperfusion injury, septic shock, side effects from radiation therapy, temporal mandibular joint disease, tumor metastasis, or an inflammatory condition resulting from strain, sprain, cartilage damage, trauma, orthopedic surgery, infection.

Filamentous fungi comprise opportunistic human fungal pathogens, such as e.g. Aspergillus fumigatus, that cause a wide range of diseases including allergic reactions and local or systemic infections. The galactosaminogalactan present in the filamentous fungi cell wall has immune-modulating properties through upregulation of IL-1RA. The inventors have shown that the IL-1RA function is required for fungal infectivity. In particular, the induction or upregulation of IL-RA increases susceptibility to fungal infection. Mice devoid of the IL-1RA gene are insensitive to infection by filamentous fungi.

Therefore, in another aspect, the present invention also relates to an inhibitor of IL-1RA for use in the treatment of human fungal diseases.

In other words, the present invention also relates to the use of an inhibitor of IL-1RA for the manufacture of a medicament intended for the treatment of human fungal diseases.

In still other words, the present invention relates to a method for the treatment of human fungal diseases in a human subject in need thereof, comprising administering an efficient amount of an inhibitor of IL-1RA.

By “human fungal diseases” it is herein referred to diseases caused by fungi, and especially filamentous fungi. The skilled person may find the complete definition and list of human fungal diseases, in particular of invasive human fungal diseases in the “Revised definitions of invasive fungal disease from the European Organization for Research and Treatment of Cancer/Invasive Fungal Infections Cooperative Group and the National Institute of Allergy and Infectious Diseases Mycoses Study Group (EORTC/MSG) Consensus Group.” (De Pauw et al.; Clin Infect Dis.; 46(12):1813-21. 2008). Human fungal diseases comprise fungus infection by any kind of fungus, Athlete's foot also known as tinea pedis (caused by fungi from the genus Trichophyton), and acute invasive pulmonary aspergillosis. Fungus infection may be diagnosed by histological analysis or culture of a specimen of tissue taken from a site of disease.

Over the past 25 years, Aspergillus fumigatus has become the most prevalent airborne fungal pathogen, causing severe and usually fatal invasive infections in immunocompromised hosts in developed countries. In a preferred embodiment of the invention, the human fungal disease of the invention is a disease caused by, or associated with, Aspergillus fumigatus. In an especially preferred embodiment of the invention, the human fungal disease is acute invasive pulmonary aspergillosis.

Inhibitors of IL-1RA, as meant herein, encompass all compounds that are capable of inhibiting the expression and/or the activity of IL-1RA.

By “inhibiting the expression of IL-1RA”, it is herein referred to the ability of a compound to reduce or annihilate the expression of the transcript IL-1RA (NCBI ref.: NM_{—}000577.4), and/or reduce or annihilate the expression of the protein IL-1RA (NCBI ref.: NP_{—}000568.1). The skilled person will clearly understand that the ability of a compound to reduce or annihilate the expression of IL-1RA can be assessed by the above-disclosed methods for measuring IL-1RA concentration at the transcript or at the protein level. Preferably, the concentrations of IL-1RA measured when the inhibitor is used will be compared to a control devoid of said inhibitor. In that case, the level of the concentrations of IL-1RA measured when the inhibitor is used is inferior or equal to those obtained with a control devoid of said inhibitor.

By “inhibiting the activity of IL-1RA”, it is herein referred to the ability of a compound to reduce or annihilate the binding of IL-1RA to the IL-1 receptor. The binding of a ligand to a receptor can easily be measured through conventional techniques that are well known in the art, such as immunohistochemistry, ELISA, western blot analysis, surface plasmon resonance (for example with the BIAcore technology), dual polarisation interferometry, and Microscale Thermophoresis (MST), as well as assays used in high throughput screening (HTS) applications, such as scintillation proximity assay (SPA). In the context of the invention, these assays can for example be performed using soluble IL-1 receptor domains, stabilization of IL-1 receptor into a membrane-like environment or direct use of solubilized IL-1 receptor. For a thorough description of such assays, the skilled person may refer to Jong et al. (J Chromatogr B Analyt Technol Biomed Life Sci.; 2005; 829(1-2):1-25).

According to the invention, inhibitors of IL-1RA comprise antibodies and polynucleotides, including antisense polynucleotides, interfering RNAs (iRNAs), and small interfering RNAs (siRNAs). According to the invention, interfering RNA are double stranded RNA molecules which are able to inhibit expression of targeted gene products. According to the invention, small interfering RNA are iRNA of 20-25 base pairs in length. iRNAs and their use in inhibiting the expression of targeted gene product are well known from the skilled person and do not need to be further explained herein. For a thorough description of iRNA, siRNA, and their use, the skilled person can refer to Cejka et al. (Clinical Science, 110, 47-58; 2006) or Tuschl et al. (Chembiochem; 2, 239-245; 2001).

Antibodies specific for an antigen can be potent inhibitors, in that they impede normal functioning of the protein, at least because of steric effect. In a particular embodiment of the invention, said inhibitor is an antibody specific for IL-1RA. Inhibitors of IL-1RA include antibodies specific for IL-1RA, such as for example the monoclonal mouse IgG2A Clone#10309 (catalog number: MAB601) from R&D system, the polyclonal goat IgG (catalog number: AF-201-NA) from R&D system.

Techniques to produce polyclonal or monoclonal antibodies specific for a polypeptide and/or for said peptide fragments are disclosed above.

In a particular embodiment of the invention, said inhibitor is a polynucleotide.

By polynucleotide, it is herein referred to any nucleic acid sequence, that is to say any polyribonucleotide or polydeoxyribonucleotide, which may be unmodified RNA or DNA or modified RNA or DNA. This includes, without limitation, single and double stranded DNA, DNA including single and double-stranded regions, single and double stranded RNA, and RNA including single and double stranded regions, hybrid molecules comprising DNA and RNA that may be single stranded or, more typically, double stranded or include single and double stranded regions. Also included are triple stranded regions comprising RNA or DNA or both RNA and DNA. Specifically included are mRNAs, cDNAs, and genomic DNAs, and any fragments, and modifications thereof.

More particularly, the agent is selected from the group consisting of: an antisense nucleic acid directed to the transcript IL-1RA (NCBI ref.: NM_{—}000577.4), a nucleic acid adapted to express such antisense, iRNA directed to the transcript IL-1RA (NCBI ref.: NM_{—}000577.4), and a nucleic acid adapted to express such iRNA.

In another particular embodiment, the present invention encompasses an isolated iRNA (e.g., siRNA) directed to the transcript IL-1RA (NCBI ref.: NM_{—}000577.4), or a nucleic acid adapted in use to express an iRNA directed to the transcript IL-1RA (NCBI ref.: NM_{—}000577.4). According to the invention, the isolated iRNA can comprise a sense strand and an antisense strand which form a duplex. Such antisense polynucleotides and iRNAs, in particular, siRNAs, can inhibit expression of the protein IL-1RA (NCBI ref.: NP_{—}000568.1).

Another object of the invention is a pharmaceutical composition comprising an inhibitor of IL-1RA, and optionally a pharmaceutically acceptable carrier.

Pharmaceutically acceptable carriers have been defined above.

In a particular embodiment of the invention, said inhibitor is an antibody specific for IL-1RA.

In an embodiment, the composition of the invention is preferably formulated for oral administration.

For oral administration, the composition of the invention is preferably formulated in the form of dosage units, such as ingestible tablets, buccal tablets, capsules, or in the form of elixirs, suspensions, syrups, wafers and the like.

Pharmaceutical compositions of the present invention may further comprise other therapeutic compounds suitable for the indication being treated. Human fungal disease can be treated with antifungal compounds.

In a particular embodiment of the invention, said pharmaceutical composition further comprises a second antifungal compound.

Antifungal compounds comprise polyene antifungals, imidazole antifungals, triazole antifungals, thiazole antifungals, allylamines and echinocandins.

According to the invention, polyene antifungals comprise amphotericin B, candicidin, filipin, hamycin, natamycin, nystatin, rimocidin.

According to the invention, imidazoles antifungals comprise bifonazole, butoconazole, clotrimazole, econazole, fenticonazole, isoconazole, ketoconazole, miconazole, omoconazole, oxiconazole, sertaconazole, sulconazole, tioconazole.

According to the invention, triazoles antifungals comprise albaconazole, fluconazole, isavuconazole, itraconazole, posaconazole, ravuconazole, terconazole, voriconazole.

According to the invention, thiazoles antifungals comprise abafungin.

According to the invention, allylamines comprise amorolfin, butenafine, naftifine, terbinafine.

According to the invention, echinocandins comprise anidulafungin, caspofungin, micafungin.

In particular, known therapeutic compounds suitable for the treatment of acute invasive pulmonary aspergillosis include steroids, voriconazole and liposomal amphotericin B.

In a particular embodiment of the invention, said pharmaceutical composition further comprises at least one compound chosen from steroids, voriconazole and liposomal amphotericin B.

The composition of the invention is particularly suited for the treatment of for use in the treatment of human fungal diseases.

Another object of the invention is thus a pharmaceutical composition comprising an inhibitor of IL-1RA according to the invention for use in the treatment of human fungal diseases.

The object of the invention is therefore also the use of a pharmaceutical composition comprising an inhibitor of IL-1RA according to the invention for the manufacture of a medicament intended for the treatment of human fungal diseases.

Another object of the invention is thus a method for the treatment of human fungal diseases in a human subject in need thereof, comprising administering an effective amount of a pharmaceutical composition comprising an inhibitor of IL-1RA according to the invention.

In a particular embodiment of the invention, the human fungal disease is acute invasive pulmonary aspergillosis.

### Experimental Results

### Methods

Extraction of Soluble Galactosaminogalactan from A. fumigatus

The A. fumigatus, strain CBS 144-89 was grown in a 15 l fermenter in modified Brian medium (2% asparagine, 5% glucose, 2.4 g/l NH4NO3, 10 g/l KH2PO4, 2 g/l MgSO4-7H2O, 26 mg/l ZnSO4-7H2O, 2.6 mg/l CuSO4-5H2O, 1.3 mg/l Co(NO3)2-6H2O, 65 mg/l CaCl2, pH 5.4) for 72 h at 25° C. The mycelium was removed by filtration under vacuum and the supernatant was precipitated with 2.5 vol. of ethanol overnight at 4° C. The pellet was collected by centrifugation (3000 g, 10 min). The pellet was washed twice with 2.5 l of 150 mM NaCl and then extracted with 8 M urea (2 h twice at room temperature under shaking). Ureasupernatants (GAG) were pooled and extensively dialyzed against water and freeze-dried. Urea-insoluble pellet (PGG) was washed with water and freeze-dried.

Soluble galactosaminogalactan (GAG) was used in a final concentration of 10 μg/ml. Before using GAG in stimulation experiments it was incubated with polymixin B to neutralize potential contamination of lipopolysaccharide.

### Stimuli and Reagents

A clinical isolate of Aspergillus fumigatus V05-27, which has been characterized previously was used for stimulations (Netea et al., 2003). Conidia and hyphae were prepared and heat-inactivated (HI) as previously described (Chai et al., 2009a). A concentration of 1×10⁷/ml was used in the experiments, unless otherwise indicated. C. albicans, strain ATCC MYA-3573 (UC820) (Gow et al., 2007; Lehrer and Cline, 1969). Candida were heat-killed (HK) at 98° C. for 1 hour and used in a final concentration of 1×107/ml. Recombinant human IL-1β, IL-23, IL-12 and IL-18 were purchased from R&D Systems (Minneapolis, Minn., USA) and were used in end concentrations of 100 ng/ml, 50 ng/ml, 10 ng/ml and 50 ng/ml respectively. Recombinant human (rh) IL-1Ra (Amgen, Inc., Thousand Oaks, Calif., USA) was used to antagonize IL-1β signalling at a final concentration of 10 ng/ml. Anti-human IL-1Ra (R&D) was used to block IL-1Ra in a final concentration of 10 μg/ml, and was compared to isotype control. Pattern recognition pathways were inhibited in PBMCs by pre-stimulation for 1 hour with a specific inhibitor. LPS derived from Bartonella quitana was used to block TLR4 (Abdollahi-Roodsaz et al., 2007). B. quitana LPS was extracted and purified as described previously (Hirschfeld et al., 1999). Mouse anti-humanTLR-2 (eBioscience, Halle-Zoersel, Belgium) and control mouse IgG1 (eBioscience) were used at a final concentration of 10 μg/mL. Anti-human integrin β2 (αCR3) and control goat IgG were purchased from R&D systems (Minneapolis, Minn., USA) and used in a final concentration of 10 μg/mL. Laminarin was kindly provided by Professor David Williams of Tennessee University and was used in a final concentration of 50 ng/mL to inhibit dectin-1. Syk kinase inhibitor was purchased from Calbiochem (Merck, Darmstadt, Germany) and was used in a concentration of 50 nM to inhibit Syk signaling.

### PBMC Isolation and Stimulation

Venous blood from healthy volunteers was drawn into 10 ml EDTA tubes after written informed consent, after which PBMCs were isolated as previously described (van de Veerdonk et al., 2009). Briefly, blood was diluted in phosphate buffered saline (PBS) (1:1) and fractions were separated by Ficoll (Ficoll-Paque Plus, GE healthcare, Zeist, The Netherlands) density gradient centrifugation. Cells were washed twice with PBS and resuspended in RPMI-1640 culture medium supplemented with 10 μg/ml gentamicin, 10 mM L-glutamine and 10 mM pyruvate (Gibco, Invitrogen, Breda, The Netherlands). The cells were counted using a particle counter (Beckmann Coulter, Woerden, The Netherlands) and the cell number was adjusted to 5×106/ml. PBMCs were plated in 96 well roundbottom plates (Corning, N.Y., USA) at a final concentration of 2.5×106/ml and in a total volume of 200 μl. Cells were prestimulated for 1 hour with medium or 10 μg/ml GAG. Following prestimulation, the PBMCs were stimulated with culture medium, HI A. fumigatus conidia (1×107/ml), IL-1β/IL-23 (100 and 50 ng/ml respectively) or IL-12/IL-18 (10 and 50 ng/ml respectively). These experiments were also performed in the presence of 10 μg/ml anti-IL-1Ra antibody or isotype control. Plates were incubated at 37° C., 5% CO2 for 24 hours, 48 hours or 7 days. 7 day cultures were supplemented with 10% human serum. After incubation, culture supernatants were collected and stored at −20° C. until cytokine measurements were performed.

### IL-1 Bioassay

The murine cell line NOB-1 responds to both human or mouse IL-1 by production of IL-2, furthermore these cells are unresponsive to other cytokines like tumor necrosis factor (TNF), colony stimulating factors (CSFS), IL-3, IL-5, IL-6 and IFNγ (Gearing et al., 1987). NOB-1 cells were plated in 96 well flatbottom plates at a final density of 1×106 cells/ml and were stimulated for 24 hour using culture supernatants of PBMCs stimulated in presence or absence of GAG (as described above). After 24 hours of incubation at 37° C., 5% CO2 the culture supernatants of the NOB-1 cells were collected and IL-2 production by the NOB-1 cells was measured by ELISA (R&D systems).

### Cytokine Measurement

Cytokines were measured using commercially available ELISAs (R&D Systems)(Sanquin, Amsterdam, The Netherlands) according to the protocols supplied by the manufacturer. IL-1β, TNF-α, IL-8 and IL-1Ra were measured in culture supernatants of 24 hour cultures, IFN-γ and IL-10 were measured in culture supernatants of 48 h hour cultures, and IL-17 and IL-22 were measured in culture supernatants of 7 day cultures.

### Mice

Female, 8- to 10-weeks old, BALB/c (wild-type-WT) mice were purchased from Charles River (Calco, Italy). Breeding pairs of homozygous Il1ra^{+/-} mice on the BALB/c background, were bred under specific-pathogen free conditions at the breeding facilities of the University of Perugia, Perugia, Italy. Experiments were performed according to the Italian Approved Animal Welfare Assurance 229-2011-B.

### Fungal Infection, Allergy and Treatment

Viable conidia from the A. fumigatus Af293 strain were obtained as described (Bonifazi et al., Mucosal Immunol 3: 193-205, 2010). For infection mice were anesthetized in a small plastic cage, containing 3% Isofluoran (Isofluran Forene Abbot Scandinavia AB, Solna) before intranasal (i.n.) instillation of a suspension of 2×10⁷ resting conidia/20 μl saline. Mice were treated with 250 μg/kg i.n. of GAG the day of infection and on days 1 to 3 post infection Mice were monitored for survival, fungal growth (colony forming unit/organ, mean±SE) as described (Bozza et al., Blood 102: 3807-3814, 2003), polymorphonuclear cells recruitment in bronchioalveolar lavage fluid (BAL), histopathology, myeloperoxidase (Mpo) and Il1ra mRNA expression in lung cells and IL-1Ra production.

For histology, sections (3-4 μm) of paraffin-embedded tissues were stained with periodic acid-Schiff (PAS) reagent. For allergy, mice received an i.p. and s.c. injection of 100 μg of A. fumigatus culture filtrate extract (CCFA) dissolved in incomplete Freund's adjuvant (Sigma) followed by two consecutive intranasal injections (a week apart) of 20 μg CCFA. A week after the last intranasal challenge, mice received 10⁷Aspergillus resting conidia and evaluated a week later (16424201). GAG (250 μg/kg i.n.) or vehicle alone was administered daily, for a week, in concomitance with the Aspergillus infection.

### Collection of Broncho-Alveolar Lavage (BAL) Fluid

Lungs were filled thoroughly with 1 ml aliquots of pyrogen-free saline through a 22-gauge bead-tipped feeding needle introduced into the trachea. The lavage fluid was collected in a plastic tube on ice and centrifuged at 400 g, 4° C., for 5 min. For differential BAL cell counts, cytospin preparations were made and stained with May-Grünwald Giemsa reagents (Sigma-Aldrich). At least 200 cells per cytospin preparation were counted and the absolute number of each cell type was calculated. Photographs were observed using a BX51 microscope (Olympus, Milan, Italy) and images were captured using a high-resolution DP71 camera (Olympus).

### Dextran Sulfate Sodium-Induced Colitis

Mice received either regular drinking water (control) or 2.5% dextran sulfate sodium (DSS) in drinking water for 7 days and then allowed to recover by drinking water alone for an additional 7 days. GAG was given intraperitoneally (1 mg/kg) daily for a week. Weight changes were recorded daily, and the day after the 7-days of rest mice were killed and tissues were collected for histology and cytokine analysis. Colonic sections were stained with H&E (Takedatsu 2008). To assess colitis severity, stool and histological scores were used that recently were introduced and proven sensitive to experimental therapy (PMID: 21763243).

### Cell Purification and Cell Cultures

Purified peritoneal CD11b⁺Gr-1⁺ polymorphonuclear neutrophils (PMNs) (>98% pure on FACS analysis) were obtained as described (Bellocchio et al., J Immunol 173: 7406-7415, 2004). Lung epithelial cells were isolated as described (You et al., Cell Mol Physiol 283: L1315-1321, 2002). Murine macrophages were isolated from total lung cells after 2 hours plastic adherence at 37° C. PMNs, epithelial cells and macrophages were exposed to unopsonized Aspergillus conidiaat the ratio of 1:1 or LPS (10 ng/ml) at 37° C. for 1 hour in the presence of different concentrations (1 or 20 μg/ml) of GAG for 18 hours before the assessment of Il1ra mRNA expression.

### Statistical Analysis

The differences between the various stimulations were analyzed with the Wilcoxon signed rank test (p-value of <0.05 was considered statistically significant). All experiments were performed at least twice and data represent cumulative results of all experiments performed and are presented as mean+/−standard error of the mean (SEM) unless otherwise indicated. Data was analyzed using GraphPad Prism v 5.0.

### Results

### Soluble Galactosaminogalactan Modulates Human T-Helper Cytokine Responses

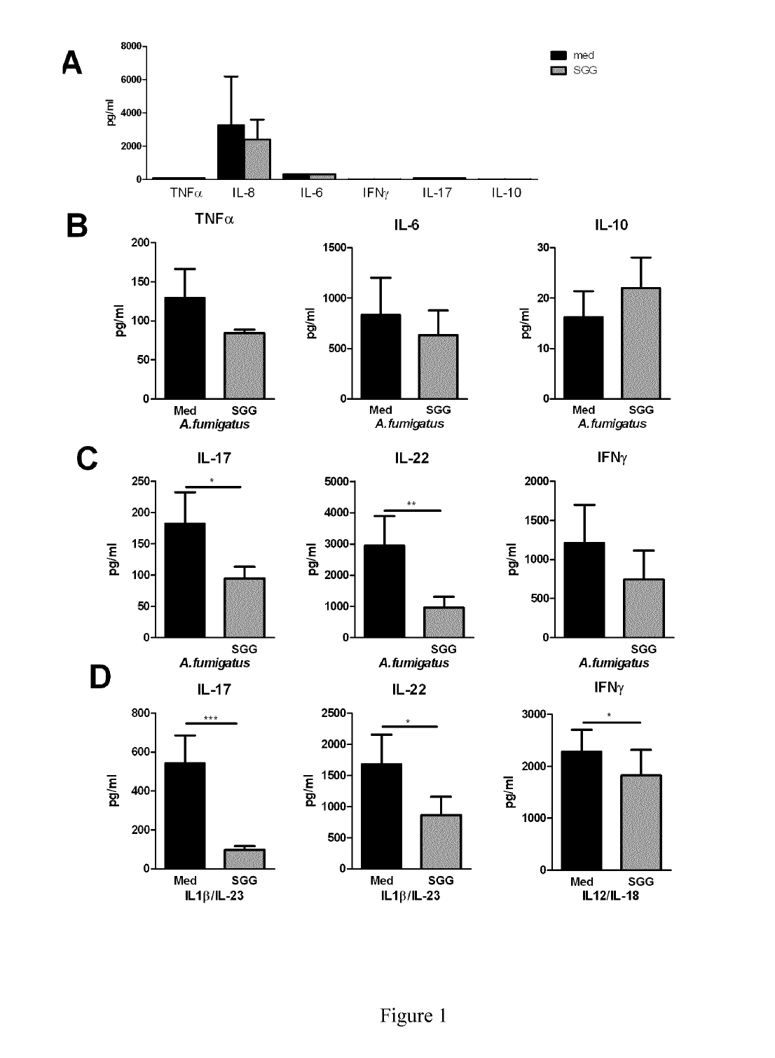

To investigate whether GAG can exert immunomodulatory effects in humans, we tested whether GAG induces the production of pro- and/or anti-inflammatory cytokines in human PBMCs. GAG did not induce the proinflammatory cytokines TNFα, IL-6, IL-8, IFNγ, and IL-17 (FIG. 1A), neither did it induce the anti-inflammatory cytokine IL-10 (FIG. 1A). To determine whether GAG modulates Aspergillus-induced innate monocyte-derived cytokines, we stimulated PBMCs for 24 hours with Aspergillus conidia in the presence or absence of GAG. The presence of GAG did not have a significant effect on the production of the innate cytokines TNFα and IL-6, or the anti-inflammatory cytokine IL-10 (FIG. 1B). However, when we investigated the production of the characteristic T-helper cytokines IL-17, IL-22 and IFNγ induced by A. fumigatus, the IL-17 and IL-22 responses were significantly reduced in the presence of GAG (FIG. 1C). To determine whether the effects of GAG are specific for Aspergillus-driven T-helper responses or that GAG has a general ability to modulate human T-helper responses, we investigated the effects of GAG on cytokine-driven T-helper responses. GAG significantly decreased the proinflammatory T-helper cytokine production induced by the cytokine combinations IL-1β/IL-23 and IL-12/IL-18 that induce IL-17/IL-22 and IFNγ respectively (FIG. 1E). Thus, GAG can inhibit human proinflammatory T-helper cytokine production induced by Aspergillus and cytokine combinations.

### Soluble Galactosaminogalactan Induces IL-1 Receptor Antagonist.

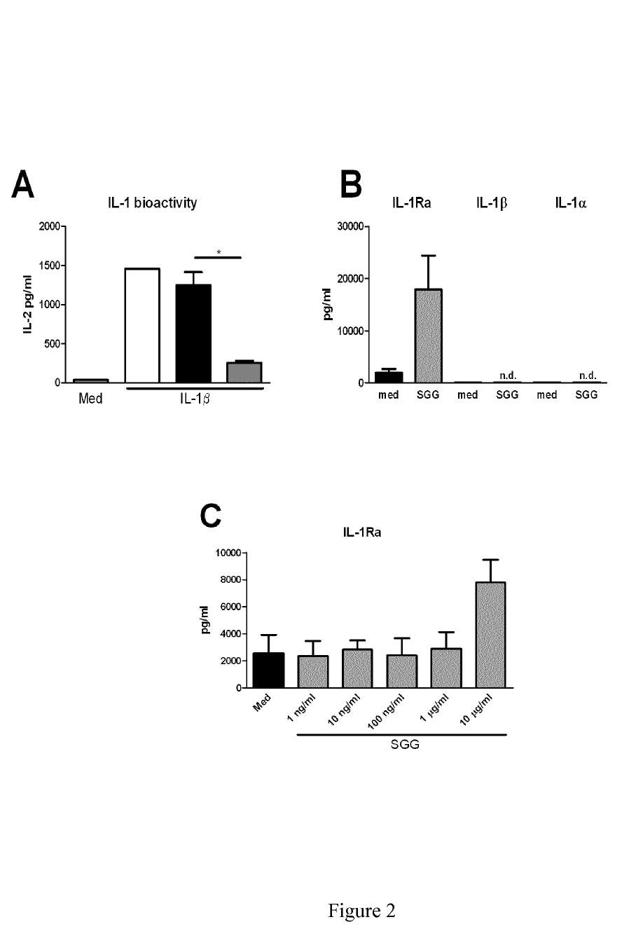

Human T-helper cytokine responses such as IL-17 and IL-22 production are highly dependent on the IL 1 receptor pathway (Ben-Sasson et al., 2009; Guo et al., 2009). We wanted to investigate whether the observed modulation of these T-helper cytokines by GAG was due to an interaction of GAG with the IL 1 pathway. Therefore, we measured the capacity of GAG conditioned medium (culture supernatants of PBMCs that were exposed to 10 μg/ml GAG for 24 hours) to reduce IL-1β bioactivity (FIG. 2A). Indeed it was shown that GAG significantly reduced the bioactivity of IL-10 while culture supernatants of unstimulated PBMCs did not (FIG. 2A). Bioactivity of the IL 1 signalling pathway is dependent on IL 1 receptor agonists (IL-1α and IL-1β) and IL 1 receptor antagonists (Dinarello, 2011). One of the natural inhibitors of the IL-1 signalling is the interleukin-1 receptor antagonist (IL-1RA), therefore we tested whether GAG can induce IL-1RA. IL-1RA concentrations in the supernatant of the cells stimulated with GAG were significantly increased compared to medium stimulated PBMCs (FIG. 2B,C), suggesting that GAG has the capacity to modulate immune responses by inducing IL-1RA. We next investigated the capacity of GAG to induce IL-1 receptor agonists, however GAG induced neither IL-1α nor IL-1β (FIG. 2B). Thus, GAG induces only IL-1 antagonistic activity without contributing to IL 1 agonistic activity.

### Suppression of IL-17 and IL-22 by Soluble Galactosaminogalactan is Dependent on IL-1RA.

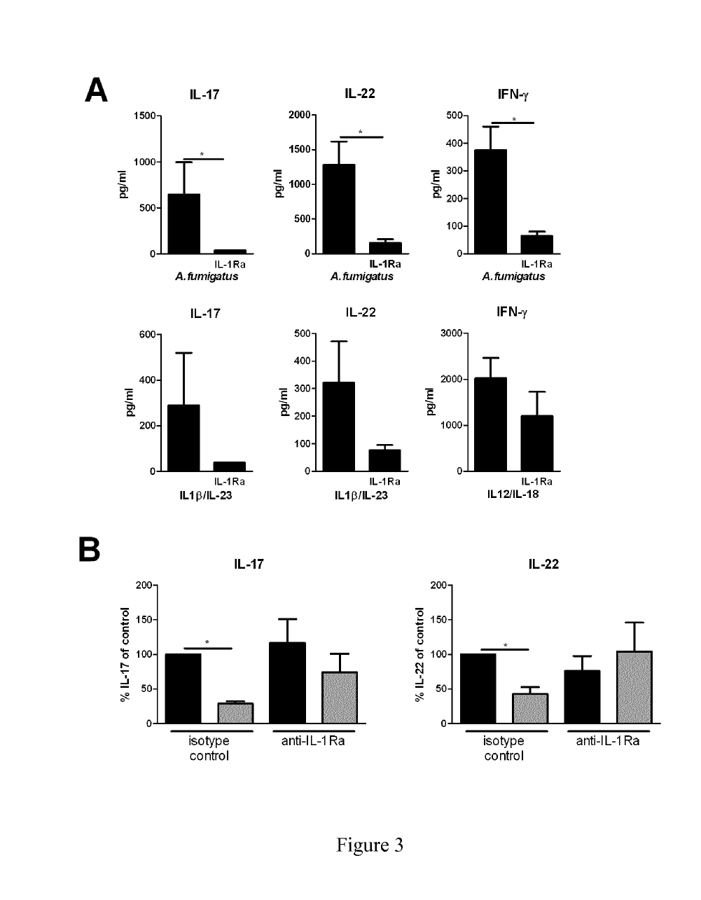

To demonstrate that IL-17, IL-22, and IFNγ production by human PBMCs is indeed dependent on the IL 1 receptor pathway and that IL-1RA can inhibit the production of these T-helper cytokines, we performed experiments of Th1 and Th17 inducing stimuli in the presence or absence of IL-1RA. Addition of IL-1RA reduced IL-17, IL-22, and IFNγ induction by both Aspergillus conidia and by IL-1/IL-23 and IL-12/IL-18 cytokine combinations (FIG. 3A). To determine whether the immunosuppressive effect of GAG was mediated through the induction of IL-1RA, we stimulated PBMCs with IL-1β/IL-23 in the presence of GAG and blocked IL-1RA with specific neutralizing antibodies. GAG reduced IL-17 and IL-22 levels significantly, which was not observed in the presence of neutralizing anti-IL-1RA antibodies, demonstrating that the inhibitory effects of GAG on T-helper cytokine production are dependent on IL-1RA (FIG. 3B).

### Soluble Galactosaminogalactan Induces IL-1 Ra In Vivo and IL-1RA Increases Susceptibility to Aspergillosis.

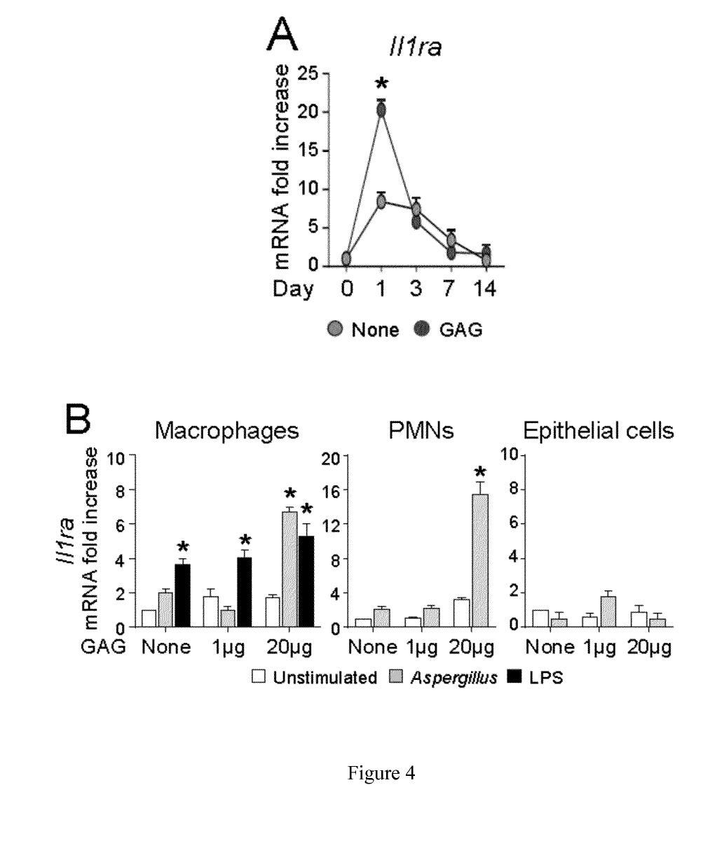

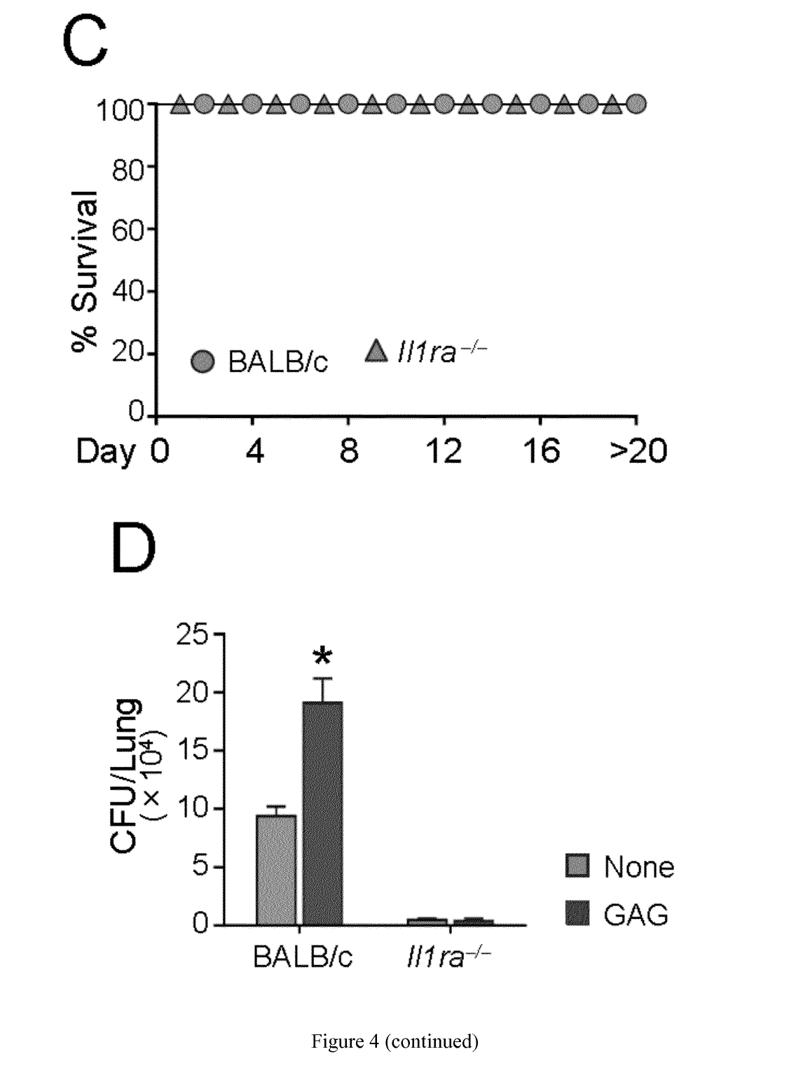

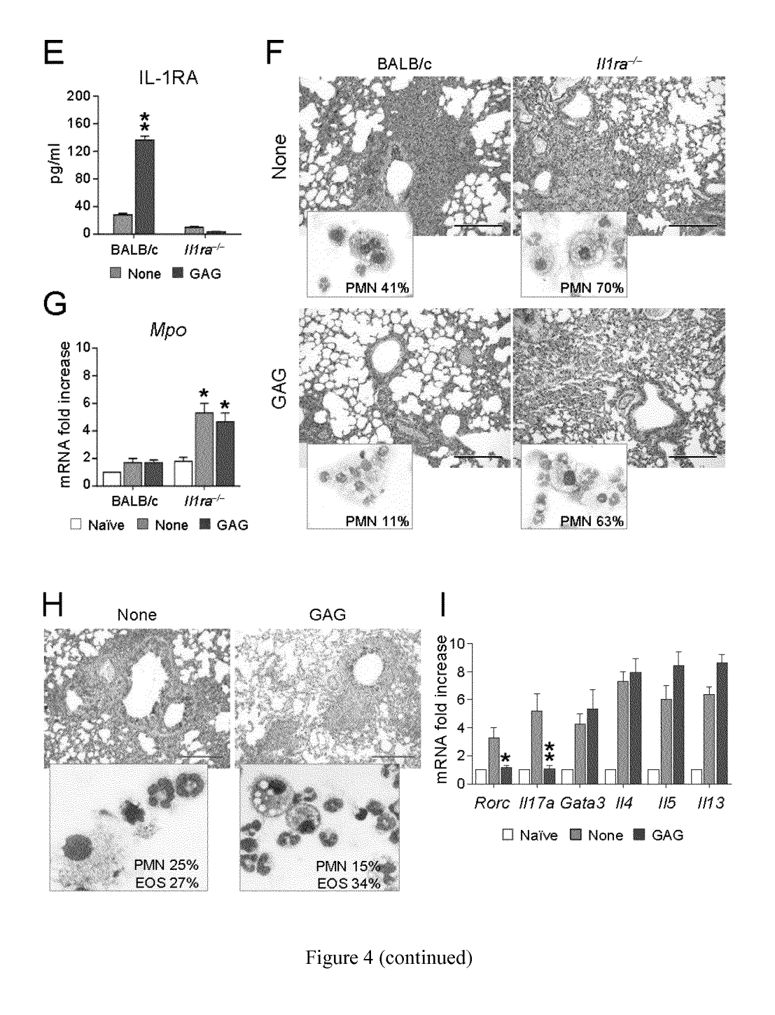

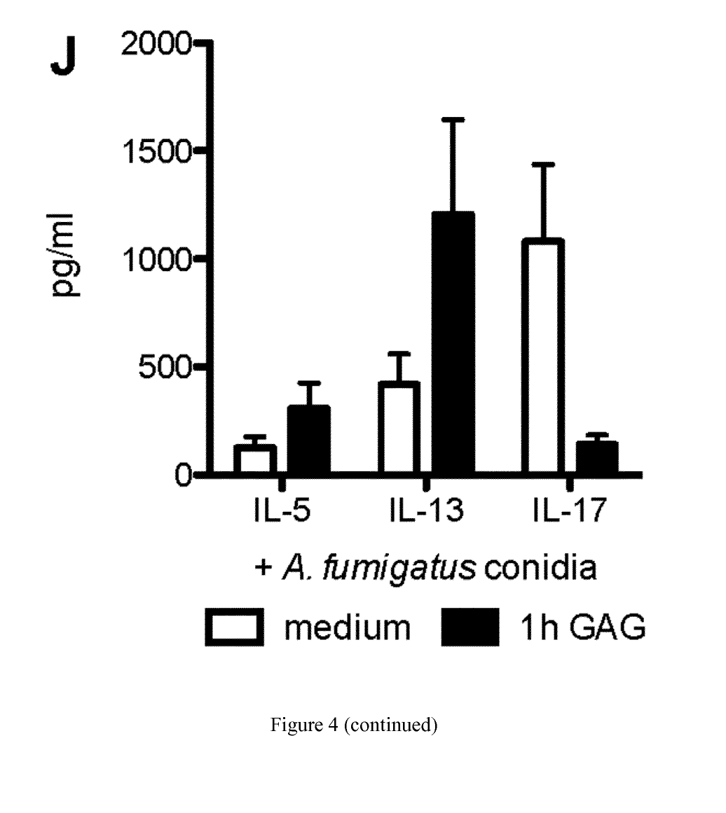

The in vitro stimulations described above suggest that the immunomodulatory effects of GAG are due to inhibition of IL-1 bioactivity by inducing IL-1RA. To assess whether this has relevant consequences in-vivo, we measured IL-1RA transcription in the lungs of mice infected with Aspergillus with or without the administration of GAG. Induction of IL-1RA was increased in the presence of GAG (FIG. 4A). To determine which cells are responsible for the induction of IL-1RA, we isolated macrophages, neutrophils and epithelial cells from the lungs of naïve mice. Macrophages and neutrophils, but not epithelial cells, induced IL-1RA after pre-stimulation with Aspergillus in the presence of GAG (FIG. 4B). Interestingly, not all microbiological stimuli can prime for increased IL-1Ra induction, since prestimulation with LPS did not increase LPS Il1ra induction by GAG (FIG. 4B).

To investigate the significance of IL-1RA in vivo and to determine whether the effects of GAG induced by GAG are dependent on IL-1RA, we studied the effects of GAG in wild type (WT) and IL-1RA^{-/-} mice with invasive aspergillosis. IL-1RA^{-/-} mice were highly resistant to invasive aspergillosis, as indicated by long-term survival (FIG. 4C) and reduced fungal load (FIG. 4D). Administration of GAG resulted in measurable increased protein levels of IL-1Ra in the lungs of wild-type mice during infection (FIG. 4E). In line with previous observations, GAG increased the susceptibility to invasive aspergillosis in WT mice but not in Il1ra^{-/-} mice (FIG. 4D). As expected, administration of GAG reduced inflammatory polymorphonuclear neutrophil (PMN) recruitment in WT mice (FIG. 4F).

However, these effects were not observed in Il1Ra^{-/-} mice. The number of PMNs in the BAL was increased in IL-1Ra^{-/-} mice (FIG. 4F) Increased PMN numbers also correlated with increased expression of myeloperoxidase (MPO) (FIG. 4G). These data demonstrate that IL-1RA has an important role in invasive aspergillosis, and support the concept that the induction of IL-1RA by GAG has important clinical consequences.

Additionally we studied the role of GAG in a model for allergic bronchopulmonary aspergillosis (ABPA). In ABPA, GAG administration decreased PMN, but not eosinophil recruitment in the BAL and lung of allergic mice (FIG. 4H), a finding consistent with decreased Th17, but not Th2 cell activation in the draining lymph nodes (FIG. 4I). To address whether GAG would have effects on Th2 responses in humans, we investigated human PBMCs which were pre-incubated for 1 h with GAG and subsequently stimulated with Aspergillus conidia for 7 days. We observed similar effects in vitro, namely IL-17 production decreased in the presence of GAG, while Th2 cytokines IL-5 and IL-13 were not decreased (FIG. 4J).

Thus, GAG has the potential to ameliorate Th17-dependent immunopathology in allergic bronchopulmonary aspergillosis.

### Induction of IL-1RA by GAG is Dependent on Syk and TLR3/TRIF.

Since the pattern recognition receptors that recognize GAG remain to be elucidated, we investigated which PRR is important for the induction of IL-1RA by GAG in human PBMCs. Blocking dectin-1 or complement receptor 3 (CR3) did not alter IL-1RA production (FIG. 5A). In addition, blocking the Toll like receptor (TLR) pathways TLR2 and TLR4, which are known to be involved in recognizing Aspergillus PAMPs (Bellocchio et al., 2004a; Bellocchio et al., 2004b; Bochud et al., 2008; Mambula et al., 2002; Meier et al., 2003), did not result in a significant reduction of IL-1RA (FIG. 5B). In contrast, inhibition of Syk demonstrated a slight, yet significant, decrease in IL-1RA induction induced by GAG, suggesting that the recognition receptor(s) involved in recognizing GAG in human cells are partially dependent on Syk signaling (FIG. 5C).

To identify the PRR pathways that are involved in the capacity of GAG to increase the susceptibility to pulmonary aspergillosis, WT, TLR2^{-/-}, TLR3^{-/-}, TLR4^{-/-}, TLR6^{-/-} TLR9^{-/-}, TRIF^{-/-} and MyD88^{-/-} mice were infected with Aspergillus with or without GAG administration (FIG. 5D). TLR3^{-/-}, TLR9^{-/-} and TRIF^{-/-} mice displayed lower fungal burden in the presence of GAG (FIG. 5D). In addition, the induction of IL-1RA induced by the administration of GAG during invasive aspergillosis was lost in TLR3^{-/-} and TRIF^{-/-} mice (FIG. 5E). Therefore, GAG is able to induce IL-1RA in vivo in a TLR3/TRIF dependent manner.

Galactosaminogalactan Protects from Experimental Colitis

The effect of GAG on experimental murine colitis was investigated and the activity of GAG was evaluated comparatively with anakinra, a known inhibitor of IL-1R (Dinarello, 2009; Dinarello et al., 2012).

Colitis was induced in C57BL/6 wild-type (WT) or p47phox^{-/-} mice that recapitulate several of the features of the human Chronic Granulomatous Disease (CGD) (Huang et al., 1998; Romani et al., 2008). Colitis is a severe complication of CGD patients, the management of which is a challenge because standard immunosuppressive therapy increases the risk of infection in already immunocompromised hosts (Huang et al., 2004). Colitis was induced with dextran sulfate sodium (DSS 2.5% in drinking water for 7 days and then allowed to recover by drinking water alone for an additional 7 days) and the effect of GAG (1 mg/kg intraperitoneally for a week) on colonic injury was assessed. The day after the 7-day of rest, mice were killed and tissues were collected for histology, RNA and cytokine analysis. Colonic sections were stained with H&E, and histology was scored. Cytokines were measured by enzyme-linked immunoabsorbent assay (ELISA) and real time-polymerase chain reaction (RT-PCR) on colonic tissues.

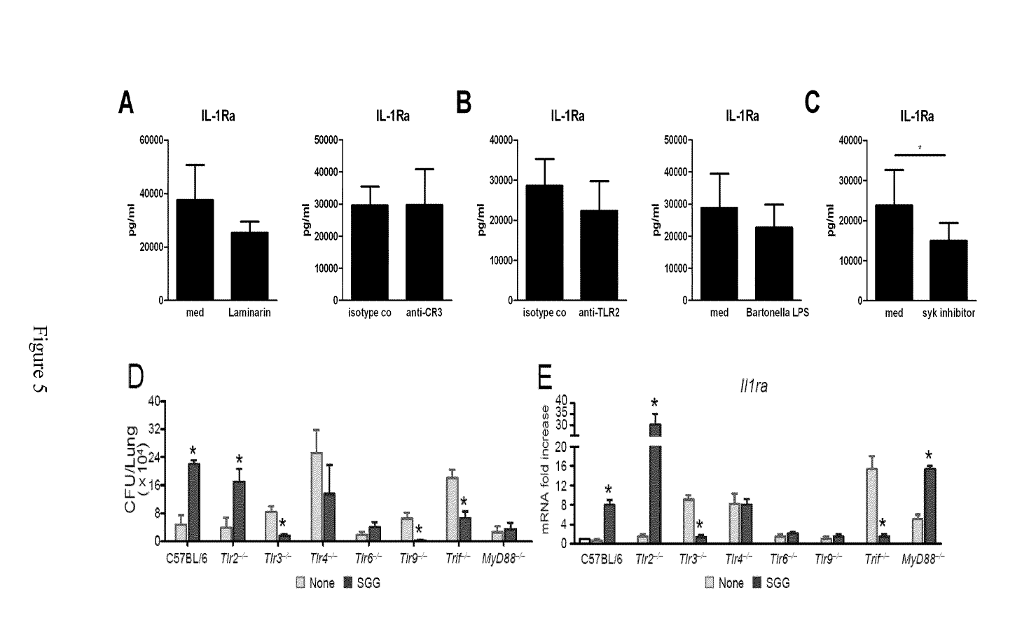

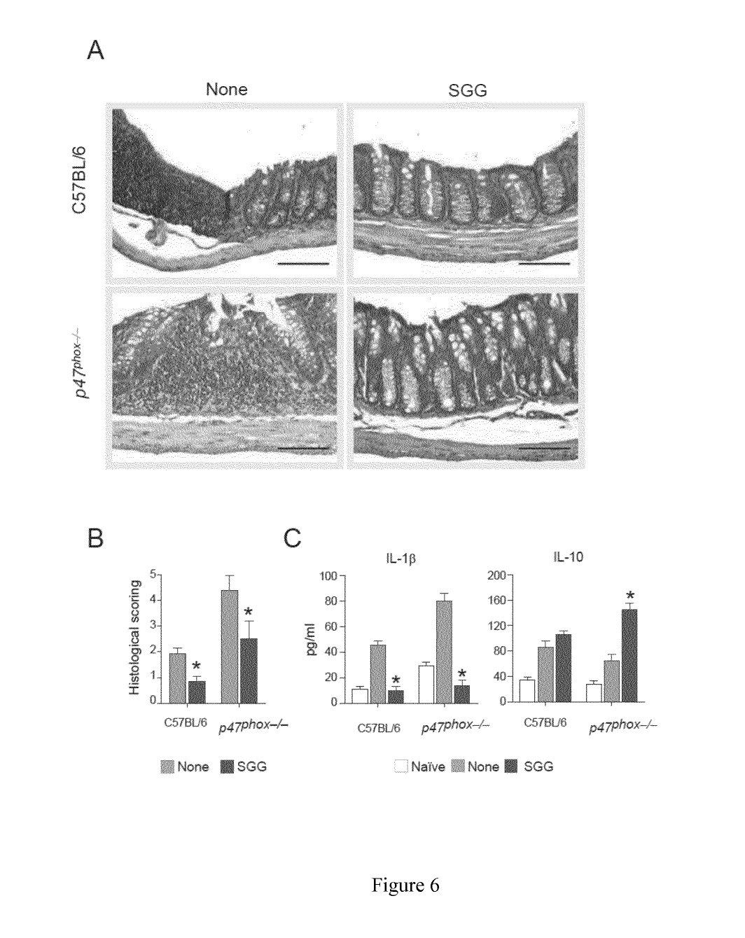

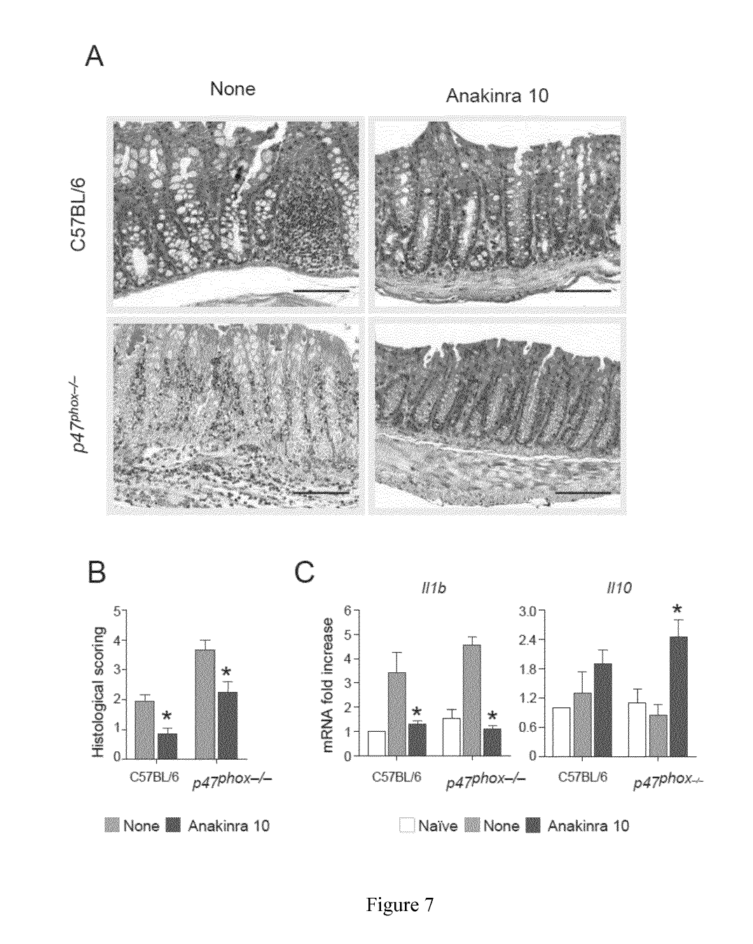

Administration of GAG significantly (*, P<0.05, treated vs untreated mice) attenuated DSS-induced colitis in WT and p47phox^{-/-} mice as observed by a significant reduction of the inflammatory colonic pathology (FIG. 6A), damage score (FIG. 6B) and inflammatory IL-1b production and a significant increase in anti-inflammatory IL-10 production (FIG. 1C). The effects was similar to that observed with anakinra, in terms of inflammatory pathology (FIG. 7A), damage score (FIG. 7B) and cytokine gene expression (FIG. 7C).

### Conclusions/Significance

Together, these results clearly demonstrated that intraperitoneally injected GAG is effective in reducing the severity of DSS-induced colitis in patients with autoinflammatory diseases, such as the CGD patients.

### The GAG Fraction Capable of Inducing IL-1RA is Composed of a Mixture of GalNAc/GalNH₂ Oligosaccharides.

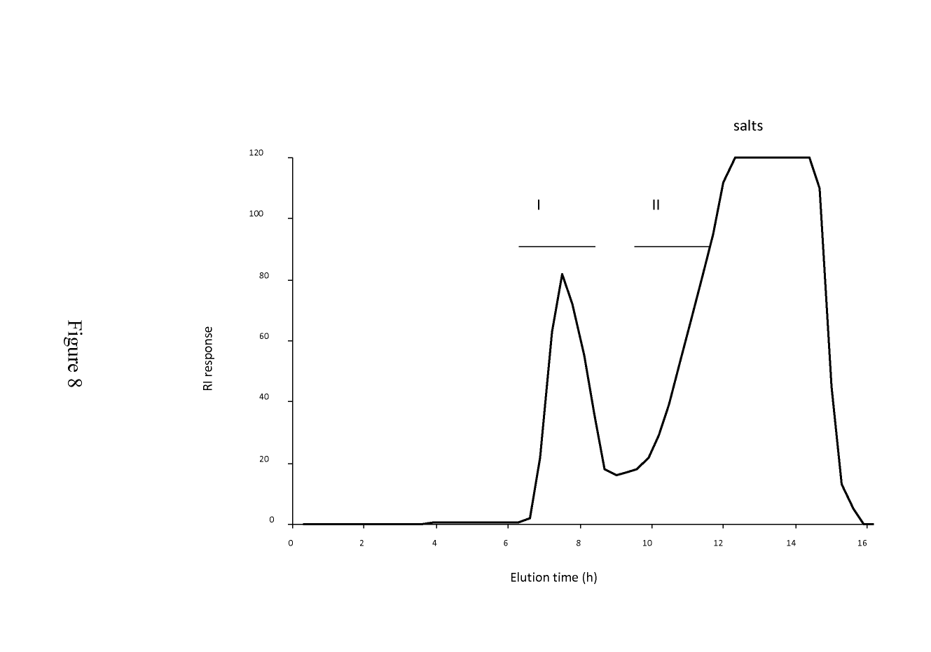

After mild acid hydrolysis, two main oligosaccharide fractions (G25I and G25 II) were isolated from the G25-Sepadex chromatography (FIG. 8). Three independent hydrolysis batches from 2 different GAG preparations were performed and resulted in similar chromatographic patterns.

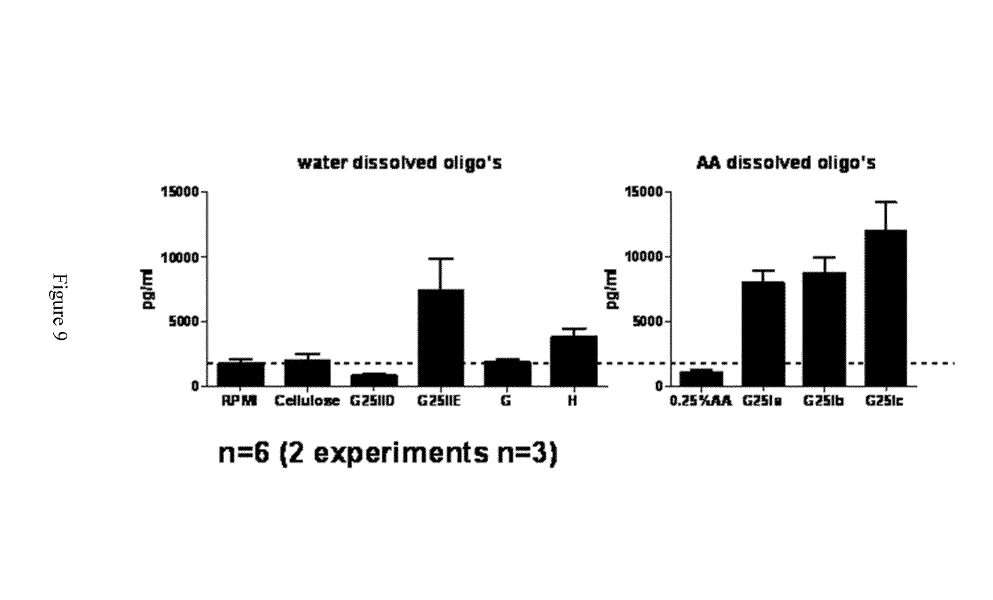

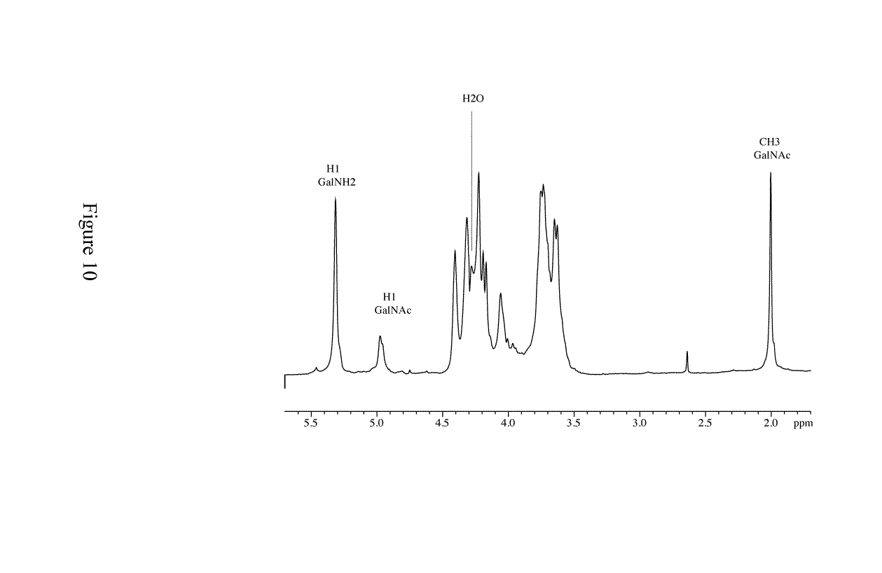

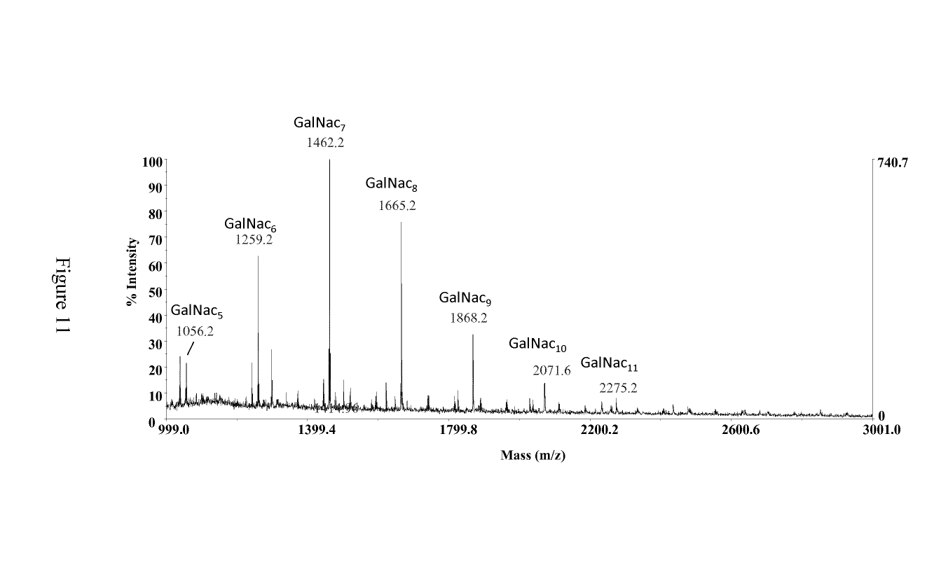

Both fractions, G25I and G25II, were tested to their putative IL-1Ra induction on PBMC. Fractions G25II did not induce significantly IL-1Ra. In contrast, all 3 batches of G25I fractions induced the production of IL-1Ra, showing that these fractions were active (FIG. 9). ¹H and ¹³C NMR analysis of these G25I fractions showed that they are composed of a mixture of GalNAc/GalNH2 oligosaccharides. (FIG. 10). MALDI-TOF analysis showed that active oligosaccharides contain at least 4 monosaccharides (FIG. 11).

Cellulose was used as a negative control. Indeed, the IL-1Ra level in presence of polysaccharide of cellulose was identical to medium control. This result suggested that the IL-1Ra is induced specifically by GAG and not by any polysaccharide.

### Discussion

In the original report describing GAG (Fontaine et al., PLoS Pathog., 7(11):e1002372, 2011), it was shown that GAG has anti-inflammatory effects in mice. However, the mechanism through which GAG elicits its immunomodulatory effects remained a question at that time. In the present study, we demonstrate that GAG induces its anti-inflammatory effects by inducing the potent anti-inflammatory cytokine IL 1 receptor antagonist.

IL-1Ra can inhibit the activation of the IL 1 pathway by binding to the IL-1R type 1 receptor and prevents recruitment of the IL-1R accessory protein that is required for signaling. It has been repeatedly shown that IL-1 is an essential proinflammatory cytokine of the innate immunity. A deficient IL 1 pathway is also detrimental for the host, since it is an important protective pathway required to fight infection (van de Veerdonk et al., Trends Immunol 32: 110-116, 2011). Thus the IL-1 axis is a potent pro-inflammatory pathway that needs to be tightly regulated, and IL-1Ra is a crucial player in this regulation. Therefore, it is rather surprising that the role of IL-1Ra in invasive fungal infection has not been studied in detail to date. We observed that the absence of IL-1Ra completely protects mice from developing invasive pulmonary aspergillosis, underscoring the importance of the IL-1 pathway in clearance of an acute invasive Aspergillus infection. The observation that GAG induces IL-1Ra in vivo identifies GAG as a potent anti-inflammatory molecule that suppresses the IL 1 pathway, subsequently resulting in increased susceptibility to invasive aspergillosis. The relevance of the IL-1 pathway in aspergillosis is underscored by the fact that polymorphisms IL-1 gene cluster polymorphisms are associated with susceptibility to develop in invasive pulmonary aspergillosis (Sainz et al., J Clin Immunol 28: 473-485, 2008), and that dectin-1 knockout mice display increased fungal burden and mortality during invasive aspergillosis, which is dependent on IL 1 (Werner et al., J Immunol 182: 4938-4946, 2009).

One of the major risk factors that increases susceptibility to invasive aspergillosis is neutropenia (Marr et al., Blood 100: 4358-4366, 2002), and neutrophils are crucial for clearing invasive germinating and hyphal forms of Aspergillus infection (Schaffner et al., J Clin Invest 69: 617-631, 1982). GAG has been shown to inhibit neutrophil recruitment to the lung, which is at least partly due to neutrophil apoptosis (Fontaine et al., PLoS Pathog., 7(11):e1002372, 2011). We observed that in the presence of GAG, IL-1Ra increased during invasive aspergillosis, which correlated with decreased PMN recruitment, and therefore increased fungal burden. In contrast, IL 1Ra^{-/-} mice displayed increased neutrophil influx when exposed to Aspergillus, which could explain the resistance of IL-1Ra^{-/-} mice for invasive aspergillosis, since they can rapidly and efficiently clear Aspergillus conidia due to their increased neutrophil response. In addition to the induction of IL-1Ra by GAG in vitro and in vivo, we observed that the inhibitory effects of GAG on the proinflammatory T-helper cytokine response in human PBMCs could be restored in the presence of a neutralizing antibody against human IL-1Ra. Furthermore, the increased susceptibility to invasive aspergillosis induced by GAG is not observed in IL-1Ra^{-/-} mice. These observations strengthen the hypothesis that the anti-inflammatory properties of GAG are dependent on IL-1Ra.

The anti-inflammatory properties of GAG were present at a concentration of 10 μg/ml, which is a relevant concentration in vivo, since Aspergillus can secrete GAG in a concentration of 50 μg/ml. The finding that antibodies against GAG are present in human serum (Loussert et al., Cell Microbiol 12: 405-410, 2010) suggests that there is an adequate exposure of GAG to trigger the immune system. In the present study we were also able to demonstrate that these antibodies do not inhibit the effect of GAG, since we observed significant effects of GAG on IL-1Ra induction and inhibition of IL-17 in the presence of human serum that contained measurable concentrations of antibodies against GAG (data not shown). The relevance of GAG is highlighted by its presence in the extracellular matrix in aspergilloma resected from patients and mice with aspergillosis (Loussert et al., Cell Microbiol 12: 405-410, 2010). It is therefore expected that GAG plays a role in the immunological synapse between host immune cell and the mycelium, not only by inducing anti-inflammatory responses through IL-1Ra but also by shielding β-glucan from recognition, which has been proposed previously (Gravelat et al., PLoS Pathog 9: e1003575, 2013).

It must be taken into account that in the setting of chronic inflammation in which neutrophils and increased Th17 responses are detrimental for the host, IL-1Ra plays a protective role, due to its significant capability to suppress the IL 1 signaling pathway. This hypothesis is in line with the observation that IL-1Ra^{-/-} mice develop spontaneous destructive arthritis that is IL 1 and Th17 dependent (Koenders et al., Arthritis Rheum 58: 3461-3470, 2008). The importance of IL-1Ra in controlling IL 1 mediated proinflammatory responses is underlined by a disease called deficiency of IL-1Ra (DIRA). This disease is characterized by the absence of IL-1Ra and severe Th17 mediated responses with neutrophil influx in the skin and bones of these patients, subsequently resulting in severe skin inflammation and osteomyelitis (Aksentijevich et al., N Engl J Med 360: 2426-2437, 2009). Therefore, the timing of IL-1Ra induction is of utmost importance to protect the host from infection and overwhelming inflammation. Since GAG induces IL-1Ra, we envisage a model in which GAG on the one hand might be detrimental for the host in the setting of an acute infection, and on the other hand could be beneficial for the host in the setting of chronic inflammation driven by IL 1.

Chronic allergic aspergillosis is associated with excessive inflammation, with increased production of IL 1 and IL-22 (Lilly et al., J Immunol. 189(7):3653-360, 2012). We have demonstrated in the present study that administration of GAG induces IL-1Ra and is able to decrease IL-22 production. Therefore we investigated the effect of GAG in a murine model of allergic bronchopulmonary aspergillosis (ABPA). We observed that the administration of GAG reduces the amount of neutrophils, but not eosinophils in ABPA. Additionally, Th17 responses were down-regulated, but not Th2 responses. It is therefore tempting to speculate that administration of GAG can be beneficial in the setting of chronic allergic inflammation that is associated with excessive neutrophil-driven inflammation by reducing Th17 dependent pathology by inhibiting the IL 1 pathway. In addition, we demonstrate that GAG protects CGD mice from DSS-induced colitis similarly to IL-1Ra. Therefore, next to the identification of GAG or IL-1Ra as a therapeutic target for invasive aspergillosis, it is the first time that a polysaccharide produced by a human pathogen has been identified as an inducer of IL-1Ra by cells of the innate immunity, and which has therapeutic capacity in IL 1 mediated disease. The search of the sensing and signal transduction cascade activated by this polysaccharide will now be the center of future research.

The data presented here brings new questions into light and opens opportunities for future research. First, one of the most interesting observations is the complete protection of IL-1Ra knockout mice to invasive pulmonary aspergillosis. This opens new treatment strategies that target IL-1Ra in the setting of an acute invasive fungal infection. Second, the significant induction of IL-1Ra by GAG makes GAG or a derivative structure of GAG a potential treatment compound for IL 1 mediated diseases, such as joint, bone and muscle diseases and even very common inflammatory diseases such as diabetes and gout (Dinarello et al., Nat Rev Drug Discov 11: 633-652, 2012). Previously, we have shown that mitogenic stimulation of monocyte derived macrophages and lymphocytes by αCD3/αCD28 coated beads, or recombinant cytokine-induced IL-17 and IFN-γ production is inhibited in the presence of live A. fumigatus (Chai et al., Immunology 130: 46-54, 2010). Although these changes in cytokine responses were attributed to changes in tryptophan and kynurenine, it is tempting to speculate that GAG secretion by live A. fumigatus is responsible for the decreased IL-17 production.

In conclusion, our results demonstrate that GAG has potent anti-inflammatory effects in mice and humans that can be explained by the capability of GAG to induce IL-1Ra. These observations help to explain one of the immune-evasive mechanisms of A. fumigatus. Moreover, inhibition of GAG or IL-1Ra might prove beneficial in the treatment of acute invasive pulmonary aspergillosis.

1. Galactosaminogalactan comprising α1-4 linked galactose, α1-4 linked N-acetylgalactosamine, and optionally α1-4 linked galactosamine, for use in the treatment of at least one inflammatory disease.

2. Galactosaminogalactan comprising α1-4 linked galactose, α1-4 linked N-acetylgalactosamine, and optionally α1-4 linked galactosamine, for use according to claim 1, wherein said galactosaminogalactan preferably comprises at least one monomer having the formula:
(GalNAc)ₙ,
wherein n is an integer comprised between 5 and 12.

3. Galactosaminogalactan comprising α1-4 linked galactose, α1-4 linked N-acetylgalactosamine, and optionally α1-4 linked galactosamine, for use according to any one of claims 1 to 2, wherein said inflammatory disease is an interleukin-1 mediated disease.

4. Galactosaminogalactan comprising α1-4 linked galactose, α1-4 linked N-acetylgalactosamine, and optionally α1-4 linked galactosamine, for use according to any one of claims 1 to 3, wherein said galactosaminogalactan induces the expression of IL-1RA.

5. Galactosaminogalactan comprising α1-4 linked galactose, α1-4 linked N-acetylgalactosamine, and optionally α1-4 linked galactosamine, for use according to any one of claims 1 to 4, wherein said galactosaminogalactan is obtained from Aspergillus fumigatus.

6. Galactosaminogalactan comprising α1-4 linked galactose, α1-4 linked N-acetylgalactosamine, and optionally α1-4 linked galactosamine, for use according to any one of claims 1 to 5, wherein said galactosaminogalactan is obtained from Aspergillus fumigatus by a process comprising an extraction step by urea.

7. Galactosaminogalactan comprising α1-4 linked galactose, α1-4 linked N-acetylgalactosamine, and optionally α1-4 linked galactosamine, for use according to any one of claims 1 to 6, wherein said galactosaminogalactan is obtained from Aspergillus fumigatus by a process comprising the following steps:
a) Culture of an Aspergillus fumigatus mycelium;
b) Alcoholic precipitation of the culture obtained from step a);
c) Incubation of the precipitate obtained from step b) in NaCl,
d) Extraction of the insoluble fraction obtained from step c) by urea.

8. Galactosaminogalactan comprising α1-4 linked galactose, α1-4 linked N-acetylgalactosamine, and optionally α1-4 linked galactosamine, for use according to any one of claims 1 to 7, wherein the said process further comprises a step of mild acid hydrolysis of the product obtained in step d).

9. Galactosaminogalactan comprising α1-4 linked galactose, α1-4 linked N-acetylgalactosamine, and optionally α1-4 linked galactosamine, for use according to any one of claims 1 to 8, wherein said inflammatory disease is chosen from acute pancreatitis, ALS, Alzheimer's disease, cachexia/anorexia, asthma, atherosclerosis, chronic fatigue syndrome, fever, insulin diabetes, glomerulonephritis, graft versus host rejection, hemorrhagic shock, hyperalgesia, inflammatory bowel diseases, inflammatory conditions of a joint, including osteoarthritis, psoriatic arthritis and rheumatoid arthritis, ischemic injury, including cerebral ischemia (e.g., brain injury as a result of trauma, epilepsy, hemorrhage or stroke, each of which may lead to neurodegeneration), lung diseases (e.g., ARDS), multiple myeloma, multiple sclerosis, myelogenous (e.g., AML and CML) and other leukemias, myopathies (e.g., muscle protein metabolism, esp. in sepsis), osteoporosis, Parkinson's disease, pain, pre-term labor, psoriasis, reperfusion injury, septic shock, side effects from radiation therapy, temporal mandibular joint disease, tumor metastasis, or an inflammatory condition resulting from strain, sprain, cartilage damage, trauma, orthopedic surgery, infection.

10. Galactosaminogalactan comprising α1-4 linked galactose, α1-4 linked N-acetylgalactosamine, and optionally α1-4 linked galactosamine, for use according to any one of claims 1 to 9, wherein said inflammatory disease is chosen from inflammatory bowel diseases.

11. Galactosaminogalactan comprising α1-4 linked galactose, α1-4 linked N-acetylgalactosamine, and optionally α1-4 linked galactosamine, for use according to any one of claims 1 to 10, wherein said inflammatory bowel disease is chosen from Crohn's disease or ulcerative colitis.

12. Galactosaminogalactan comprising α1-4 linked galactose, α1-4 linked N-acetylgalactosamine, and optionally α1-4 linked galactosamine, for use according to any one of claims 1 to 11, wherein said inflammatory disease is chosen from rheumatoid arthritis, osteoarthritis, and other inflammatory conditions resulting from strain, sprain, trauma, infection, cartilage damage or orthopedic surgery.

13. Galactosaminogalactan comprising α1-4 linked galactose, α1-4 linked N-acetylgalactosamine, and optionally α1-4 linked galactosamine, for use according to any one of claims 1 to 12, wherein said inflammatory disease is chosen from inflammatory joint disease, multiple sclerosis, leukemia, ischemic injury, or reperfusion injury.

14. A pharmaceutical composition comprising a galactosaminogalactan comprising α1-4 linked galactose, α1-4 linked N-acetylgalactosamine, and optionally α1-4 linked galactosamine, as defined in any one of claims 1 to 8, and optionally a pharmaceutically acceptable carrier.

15. Pharmaceutical composition according to claim 14, wherein it further comprises a second anti-inflammatory compound chosen from non-steroidal anti-inflammatory drugs (NSAIDs), corticosteroids, slow acting anti-rheumatic drugs (SAARDs), or disease-modifying antiarthritic drugs (DMAARDs).

16. Pharmaceutical composition according to any one of claim 14 or 15, for use in the treatment of at least one inflammatory disease.

17. Pharmaceutical composition according to any one of claim 14 or 15 for use according to claim 14, wherein said inflammatory disease is an interleukin-1 mediated disease.

18. Pharmaceutical composition according to any one of claim 14 or 15 for use according to claim 14 or 15, wherein said inflammatory disease is chosen from acute pancreatitis, ALS, Alzheimer's disease, cachexia/anorexia, asthma, atherosclerosis, chronic fatigue syndrome, fever, insulin diabetes, glomerulonephritis, graft versus host rejection, hemorrhagic shock, hyperalgesia, inflammatory bowel diseases, inflammatory conditions of a joint, including osteoarthritis, psoriatic arthritis and rheumatoid arthritis, ischemic injury, including cerebral ischemia (e.g., brain injury as a result of trauma, epilepsy, hemorrhage or stroke, each of which may lead to neurodegeneration), lung diseases (e.g., ARDS), multiple myeloma, multiple sclerosis, myelogenous (e.g., AML and CML) and other leukemias, myopathies (e.g., muscle protein metabolism, esp. in sepsis), osteoporosis, Parkinson's disease, pain, pre-term labor, psoriasis, reperfusion injury, septic shock, side effects from radiation therapy, temporal mandibular joint disease, tumor metastasis, or an inflammatory condition resulting from strain, sprain, cartilage damage, trauma, orthopedic surgery, infection.
